SEQUENCE LISTING (18 sequences)

SEQ ID NO: 1 - protein, 365 aa, synthetic construct
  REGION 1..21: note = CD8 leader
  REGION 24..136: note = SpyCatcher
  REGION 139..183: note = CD8a hinge
  TRANSMEM 201..311: note = CD8a transmembrane domain
  REGION 314..365: note = CD3z intracellular signalling domain
MALPVTALLLPLALLLHAARPGSVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - DNA, 1098 bp, synthetic construct
  source 1..1098: mol_type = other DNA
atggccttaccagtgaccgccttgctcctgccgctggccttgctgctccacgccgccaggccgggatccgtgacaacactgagcggactgtctggcgagcaaggcccttctggcgatatgaccaccgaagaggatagcgccacacacatcaagttcagcaagcgcgacgaggacggcagagaacttgctggcgctaccatggaactgagagacagcagcggcaagaccatcagcacctggatctctgacggccacgtgaaggacttctatctgtaccccggcaagtacaccttcgtggaaaccgccgctcctgacggctacgaagtggccacacctatcgagttcaccgtgaacgaggatggccaagtgaccgtggatggcgaagctacagaaggcgacgcccatacagctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 3 - protein, 495 aa, synthetic construct
  SIGNAL 1..19: note = N-terminal signal sequence
  REGION 140..469: note = antibody constant domain
  MOTIF 480..495: note = SpyTag
MAWSWVFLFFLSVTTGVHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGVPHIVMVDAYKRYK

SEQ ID NO: 4 - protein, 495 aa, synthetic construct
  SIGNAL 1..19: note = N-terminal signal sequence
  REGION 140..469: note = short half life antibody constant domain
  MOTIF 480..495: note = SpyTag
MAWSWVFLFFLSVTTGVHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMASRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGVPHIVMVDAYKRYK

SEQ ID NO: 5 - protein, 476 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGVPHIVMVDAYKRYK

SEQ ID NO: 6 - protein, 476 aa, synthetic construct
  REGION 121..450: note = short half life antibody constant domain
  MOTIF 461..476: note = SpyTag
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMASRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGVPHIVMVDAYKRYK

SEQ ID NO: 7 - protein, 465 aa, synthetic construct
MAWSWVFLFFLSVTTGVHSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 491 aa, synthetic construct
  REGION 1..465: note = EGFRvIII Heavy chain
  MOTIF 476..491: note = SpyTag
MAWSWVFLFFLSVTTGVHSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGVPHIVMVDAYKRYK

SEQ ID NO: 9 - protein, 233 aa, synthetic construct
MAWSWVFLFFLSVTTGVHSDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 259 aa, synthetic construct
  REGION 1..233: note = EGFRvIII Light chain
  MOTIF 244..259: note = SpyTag
MAWSWVFLFFLSVTTGVHSDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRGVPHIVMVDAYKRYK

SEQ ID NO: 11 - protein, 468 aa, synthetic construct
MAWSWVFLFFLSVTTGVHSQVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 494 aa, synthetic construct
  REGION 1..468: note = IL13Ra2 Heavy chain
  MOTIF 480..494: note = SpyTag
MAWSWVFLFFLSVTTGVHSQVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRGVPHIVMVDAYKRYK

SEQ ID NO: 13 - protein, 237 aa, synthetic construct
MAWSWVFLFFLSVTTGVHSDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 263 aa, synthetic construct
  REGION 1..237: note = IL13Ra2 Light chain
  MOTIF 248..263: note = SpyTag
MAWSWVFLFFLSVTTGVHSDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRGVPHIVMVDAYKRYK

SEQ ID NO: 15 - protein, 468 aa, synthetic construct
MAWSWVFLFFLSVTTGVHSEVQLVESGGGLVQPGRSLRLSCAASGFTFDDYAMHWVRQAPGKGLEWVSGIGWSGGSIVYADSVKGRFTISRDNAKNSLYLQMNSLRAEDTALYYCAKDSPYGDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 265 aa, synthetic construct
  REGION 1..239: note = CD33 Light chain
  MOTIF 250..265: note = SpyTag
MAWSWVFLFFLSVTTGVHSDIVMTQSPDSLAVSLGERATINCKSSQTVFYSSNNKNYLAWYQQKPGQPPKLLISWASTRKSGVPDRFSGSGSGTDFTLTVSSLQAEDVAVYYCQHYYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRGVPHIVMVDAYKRYK

SEQ ID NO: 17 - protein, 471 aa, synthetic construct
MAWSWVFLFFLSVTTGVHSEVQLVQSGAEVKKPGASVKVSCKASGYSFTDYYMHWVRQAPGQGLEWIGRINPYAGAAFYSQNFKDRVTLTVDTSTSTAYLELSSLRSEDTAVYYCAIERGADLEGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 263 aa, synthetic construct
  REGION 1..237: note = CLL-1 Light chain
  MOTIF 248..263: note = SpyTag
MAWSWVFLFFLSVTTGVHSDIQMTQSPSSLSASVGDRVTITCRASQSVSTSSYNYMHWYQQKPGKPPKLLIKYASNLESGVPSRFSGSGSGTDFTLTISSLQPEDFATYYCQHSWEIPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRGVPHIVMVDAYKRYK

(19) United States
(12) Patent Application Publication
(10) Pub. No.: US 2024/0366665 A1
(43) Pub. Date: Nov. 7, 2024

(54) Universal Receptor Immune Cell Therapy

(71) Applicant: Prescient Therapeutics Ltd, South Melbourne (AU)

(72) Inventors: Steven L. Yatomi-Clarke, South Melbourne (AU); Rebecca Lim, South Melbourne (AU); Philip K. Darcy, South Melbourne (AU); Daniel A. Shelly, South Melbourne (AU); Kevin Sek, South Melbourne (AU)

(21) Appl. No.: 18/292,290

(22) PCT Filed: Jul. 28, 2022
(86) PCT No.: PCT/AU2022/050795

§ 371 Date: Jan. 25, 2024

(30) Foreign Application Priority Data
Jul. 28, 2021 (AU) 2021902320

(51) Int. Cl. A61K 35/17; A61K 39/00; A61P 35/00; C07K 14/705; C07K 16/28; C07K 16/32

(52) U.S. Cl. CPC A61K 35/17; A61K 39/4611; A61K 39/4613; A61K 39/4631; A61K 39/464404; A61K 39/464406; A61K 39/464419; A61K 39/464429; A61P 35/00; C07K 14/705; C07K 16/2803; C07K 16/2851; C07K 16/2863; C07K 16/2866; C07K 16/32; A61K 2239/13; A61K 2239/26; A61K 2239/38; C07K 2317/94; C07K 2319/02; C07K 2319/03

(57) ABSTRACT

The present invention relates to methods for universal immune receptor cell based therapies.

The present application claims priority from AU 2021902320, filed 28 Jul. 2021, the entire contents of which is incorporated herein by reference.

### FIELD OF THE INVENTION

The present invention relates to methods for universal immune receptor cell based therapies.

### BACKGROUND OF THE INVENTION

Despite the success of chimeric immune based cell therapies such as chimeric antigen receptor (CAR) T cells as a cellular immunotherapy for blood cancers, these therapies can have unique complications that can arise which may limit therapeutic efficacy. Major toxicities can range from treatable B cell aplasia to more severe cytokine release syndrome and neurotoxicity. Furthermore, conventional immune cell therapy has thus far shown limited efficacy to treat solid tumours. This is due to a number of factors that include the immunosuppressive tumour microenvironment (TME), inefficient cell trafficking and antigen expression heterogeneity. In the context of both solid and haematological malignancies, relapse is common due to tumour escape. These are all current unmet needs in cell immunotherapy which can be addressed through the universal immune receptor expressing immune cells.

Universal immune receptors (UIR) are composed of two discrete components, (i) the standard intracellular cell signalling domains similar to conventional CARs with an extracellular adaptor protein (such as SpyCatcher) and (ii) targeting antibodies conjugated to an adaptor protein (such as SpyTAG) (WO 2017/112784). The targeting antibody can then act as an immunologic bridge to target tumour antigens and the extracellular adaptor on the SpyCatcher receptor, eliciting an antigen specific cell response. By decoupling the antigen recognition domain of the CAR from the intracellular signalling domains into discrete components, it is possible to then target one or more multiple different antigens with the same UIR to overcome immune escape or tumour heterogeneity, to perform dose adjustment or withdrawal to regulate UIR immune cell function post administration further increasing the safety of adoptive immunotherapies.

There is a need for improved universal immune receptor cell based therapies to more effectively and reliably treat diseases such as cancers, whilst minimizing the common problems encountered with current, for example CAR T, therapies.

### SUMMARY OF THE INVENTION

The present invention provides methods for effectively treating a subject with a universal immune receptor system. The persistence of recombinant immune cells, such as CAR T cells, can be achieved through transient rest, which results in epigenetic remodelling. In the case of the present invention, this is achieved through periodic dosing and cessation of dosing of tagged binders.

In an aspect, the present invention provides a method of treating a disease in a subject that would benefit from an immune cell therapy, the method comprising
i) administering immune cells comprising a universal immune receptor to the subject, wherein the universal immune receptor may or may not be covalently bound to a molecule which comprises a domain which binds an antigen associated with the disease,
ii) administering the molecule to the subject at least twice within seven days following step i),
iii) at least about 21 days following step ii) analysing the subject for responsiveness to the treatment, and
iv) repeating steps i) and ii) if the subject has been responsive to the treatment but the disease is still detectable.

In another aspect, the present invention provides a method of stimulating a universal immune receptor mediated immune response to a tumour in a subject, the method comprising
i) administering immune cells comprising a universal immune receptor to the subject, wherein the universal immune receptor may or may not be covalently bound to a molecule which comprises a domain which binds an antigen associated with the tumour,
ii) administering the molecule to the subject at least twice within seven days following step i),
iii) at least about 21 days following step ii) analysing the subject for responsiveness to the treatment, and
iv) repeating steps i) and ii) if the subject has been responsive to the treatment but the tumour is still detectable.

In an embodiment, the molecule is not bound to the universal immune receptor in step i).

In an embodiment, the molecule is bound to the universal immune receptor in step i).

In an embodiment, in step ii) the molecule is administered twice. In an embodiment, the molecule is administered on days 3 and 6 following step i).

In an embodiment, in step ii) the molecule is administered three times. In an embodiment, the molecule is administered on days 1, 4 and 6 following step i).

In an embodiment, between about 21 days and about 49 days following step ii), the subject is analysed for responsiveness to the treatment. In an embodiment, about 21 days following step ii) the subject is analysed for responsiveness to the treatment. In an embodiment, about 49 days following step ii), the subject is analysed for responsiveness to the treatment.

In an embodiment, step ii) comprises administering the molecule to the subject at least once prior to step i) and at least twice within seven days following step i). In another embodiment, step ii) comprises administering the molecule to the subject twice prior to step i) and at least twice within seven days following step i).

In an embodiment, step (ii) comprises one of the following dosing regimens:
(a) dosing within a 24-48 hour window, and optionally repeating, within seven days following administration of the immune cells of step i);
(b) dosing every 24 hours, within seven days following administration of the immune cells of step i);
(c) periodic dosing comprising pausing treatment in a given window, resuming treatment in a next window and pausing treatment in the next window, within seven days following administration of the immune cells of step i).

In any of (a), (b) or (c) above, the UIR cells administered in step i) may be unarmed or prearmed.

In another embodiment, the molecule is administered at different doses. In an example, the different doses are a dose of about 0.75 mg/m², a dose of about 15 mg/m², and a dose of about 75 mg/m². In another embodiment the different doses may comprise any one or more of about 0.25 mg/m², about 0.5 mg/m², about 0.75 mg/m², about 1 mg/m², about 2.5 mg/m², about 5.0 mg/m², about 7.5 mg/m², about 10 mg/m², about 12.5 mg/m², about 15.0 mg/m², about 17.5 mg/m², about 20 mg/m², about 22.5 mg/m², about 25 mg/m², about 35 mg/m², about 45 mg/m², about 55 mg/m², about 65 mg/m², about 70 mg/m², about 75 mg/m², about 80 mg/m², about 85 mg/m², about 90 mg/m², about 95 mg/m² or higher. In another embodiment, the dose may be between about 0.25 mg/m²-2.0 mg/m², about 0.5 mg/m²-1.5 mg/m², or about 0.5 mg/m²-1.0 mg/m². In another embodiment, the dose may be between about 5 mg/m²-25 mg/m², between about 10 mg/m²-20 mg/m², or between about 12 mg/m²-17 mg/m². In another embodiment, the dose may be between about 50 mg/m²-100 mg/m², between about 60 mg/m²-90 mg/m², or between about 70 mg/m²-80 mg/m².

In another embodiment, the different doses of molecule administered to the subject comprise about 0.25 mg, about 24 mg or about 120 mg for a body surface area (BSA) of about 1.6. In another example, for a BSA of about 1.6, different doses of molecule administered to the subject comprise any one of about 0.1 mg, about 0.15 mg, about 0.25 mg, about 0.35 mg, about 0.45 mg, about 0.6 mg, about 12 mg, about 16 mg, about 20 mg, about 24 mg, about 28 mg, about 32 mg, about 36 mg, about 80 mg, about 90 mg, about 100 mg, about 110 mg, about 120 mg, about 130 mg, about 140 mg, about 150 mg or higher. In another embodiment, the different dose of molecule administered to the subject comprises between about 0.1 mg-2.0 mg, between about 0.2 mg-1.5 mg, or between about 0.2 mg-0.75 mg. In another embodiment, the different dose of molecule administered to the subject comprises between about 4 mg-36 mg, between about 12 mg-32 mg, or between about 20 mg-28 mg. In another embodiment, the different dose of molecule comprises between about 60 mg-160 mg, between about 80 mg-140 mg, or between about 100 mg-130 mg.

In an embodiment, step iv) comprises administering a universal immune receptor which may or may not be covalently bound to a molecule which comprises a domain which binds the same antigen as the molecule of step i). In another embodiment, step iv) comprises administering a universal immune receptor which may or may not be covalently bound to a molecule which comprises a domain which binds a different antigen to the molecule of step i).

In an embodiment, the molecule comprises a domain which binds more than one antigen associated with the disease. In an embodiment, the molecule comprises a domain which binds two antigens associated with the disease, preferably cancer. In another embodiment, the molecule comprises a domain which binds three antigens associated with the disease, preferably cancer.

In another aspect, the present invention provides a method of treating a disease in a subject that would benefit from an immune cell therapy the method comprising
i) administering immune cells comprising a universal immune receptor to the subject, wherein the universal immune receptor may or may not be covalently bound to a molecule which comprises a domain which binds an antigen associated with the disease,
ii) administering the molecule to the subject every two or three days for between about 14 days and about 28 days following step i), and
iii) repeating steps i) and ii) if the subject has been responsive to the treatment but the disease is still detectable.

In another aspect, the present invention provides a method of stimulating a universal immune receptor mediated immune response to a tumour in a subject, the method comprising
i) administering immune cells comprising a universal immune receptor to the subject, wherein the universal immune receptor may or may not be covalently bound to a molecule which comprises a domain which binds an antigen associated with the tumour,
ii) administering the molecule to the subject every two or three days for between about 14 days and about 28 days following step i), and
iii) repeating steps i) and ii) if the subject has been responsive to the treatment but the tumour is still detectable.

In an embodiment of the above aspect, the molecule is not bound to the universal immune receptor in step i).

In an embodiment of the above aspect, the molecule is bound to the universal immune receptor in step i).

In an embodiment of the above aspect, the molecule is administered every three days following step i).

In an embodiment of the above aspect, the molecule is administered every three days for about 21 days following step i).

In an embodiment of the above aspect, the subject is analysed for responsiveness to the treatment within 7 days, within 5 days, within 3 days or within a day of the completion of step ii).

In an embodiment, step ii) comprises administering the molecule to the subject at least once prior to the administration of the immune cells of step i) and every two or three days for between about 14 days and about 28 days following step i). In an embodiment, step ii) comprises administering the molecule to the subject twice prior to step i) and every two or three days for between about 14 days and about 28 days following step i).

In an embodiment, stimulating a universal immune receptor mediated immune response to a tumour comprises increasing cytokine levels in the subject, preferably increasing levels of one or more or all of interferon-γ (IFN-γ), tumour necrosis factor (TNF) and interleukin-2 (IL-2).

In an embodiment, step iii) comprises administering a universal immune receptor which may or may not be covalently bound to a molecule which comprises a domain which binds the same antigen as the molecule of step i). In another embodiment, step iii) comprises administering a universal immune receptor which may or may not be covalently bound to a molecule which comprises a domain which binds a different antigen as the molecule of step i).

In an embodiment, the molecule comprises a domain which binds more than one antigen associated with the disease. In an embodiment, the molecule comprises a domain which binds two antigens associated with the disease. In another embodiment, the molecule comprises a domain which binds three antigens associated with the disease.

In an embodiment, the treatment increases survival in the subject. In an embodiment, survival is increased when compared to a subject not receiving the treatment. In an embodiment, survival is increased by 3, 6, 9, 12, 24, 36, 48, 60, 72, 84, 96 months or more when compared to a subject not receiving the treatment.

In an embodiment, the subject has been diagnosed as having, or is suspected of having a disease such as cancer, infection or an inflammatory disease. Thus, in an embodiment, the methods described herein comprise a step of diagnosing the subject as having or suspected of having a disease such as cancer, infection or an inflammatory disease.

In an embodiment, the methods or uses further comprise the administration of an additional therapeutic agent, optionally selected from the group consisting of chemotherapy, radiotherapy, surgery, bone marrow transplant, drug therapy, cryoablation or radiofrequency ablation.

In an embodiment, the universal immune receptor comprises a SpyCatcher or a Spy Tag extracellular binding domain bound to an extracellular hinge region, which is in turn bound to a transmembrane domain which is in turn bound to an immune cell receptor intracellular signaling domain.

In an embodiment, the universal immune receptor intracellular signaling domain further comprises a costimulatory molecule.

In an embodiment, the Spy Catcher extracellular binding domain is bound to the extracellular hinge domain. In an alternate embodiment, the SpyTag extracellular binding domain is bound to the extracellular hinge domain.

In an embodiment, the molecule comprises a SpyCatcher or a SpyTag and the domain.

In an embodiment, the domain is a selected from the group consisting of an antibody, an antibody fragment, a scFv, a protein scaffold, a peptide, a ligand, an oligonucleotide, an aptamer, a tumour antigen, a self-antigen, a viral antigen, and any combination thereof. In an embodiment, the domain is an antibody or an antibody fragment.

In an embodiment, the molecule comprises SpyTag. In an alternate embodiment, the molecule comprises SpyCatcher.

In an alternate embodiment, a SpyTyg mentioned above is a SnoopTag, and a Spy Catcher mentioned above is a SnoopCatcher.

In an embodiment, the molecule is a polypeptide comprising a first domain that binds the extracellular binding domain and a second domain which binds an antigen associated with a disease. In an embodiment, the molecule further comprises a third domain which is a labelling agent.

In an embodiment, the molecule is administered to the subject at a dose of about 0.25 mg/m², about 0.5 mg/m², about 0.75 mg/m², about 1 mg/m², about 2.5 mg/m², about 5.0 mg/m², about 7.5 mg/m², about 10 mg/m², about 12.5 mg/m², about 15.0 mg/m², about 17.5 mg/m², about 20 mg/m², about 22.5 mg/m², about 25 mg/m², about 35 mg/m², about 45 mg/m², about 55 mg/m², about 65 mg/m², about 70 mg/m², about 75 mg/m², about 80 mg/m², about 85 mg/m², about 90 mg/m², about 95 mg/m² or higher. In another embodiment, the dose of molecule administered to the subject is between about 0.25 mg/m²-2.0 mg/m², between about 0.5 mg/m²-1.5 mg/m², or between about 0.5 mg/m²-1.0 mg/m². In another embodiment, the dose of molecule administered to the subject is between about 5 mg/m²-25 mg/m², between about 10 mg/m²-20 mg/m², or between about 12 mg/m²-17 mg/m². In another embodiment, the dose of molecule administered to the subject is between about 50 mg/m²-100 mg/m², between about 60 mg/m²-90 mg/m², or between about 70 mg/m²-80 mg/m². Preferably, the dose of molecule is administered to the subject is about 0.75 mg/m², about 15 mg/m² or about 75 mg/m².

In an embodiment, for a body surface area (BSA) of about 1.6, the molecule is administered to the subject at a dose of about 0.1 mg, about 0.15 mg, about 0.25 mg, about 0.35 mg, about 0.45 mg, about 0.6 mg, about 12 mg, about 16 mg, about 20 mg, about 24 mg, about 28 mg, about 32 mg, about 36 mg, about 80 mg, about 90 mg, about 100 mg, about 110 mg, about 120 mg, about 130 mg, about 140 mg, about 150 mg or higher. In another embodiment, the dose of molecule administered to the subject is between about 0.1 mg-2.0 mg, between about 0.2 mg-1.5 mg, or between about 0.2 mg-0.75 mg. In another embodiment, the dose of molecule administered to the subject is between about 4 mg-36 mg, between about 12 mg-32 mg, or between about 20 mg-28 mg. In another embodiment, the dose of molecule administered to the subject is between about 60 mg-160 mg, between about 80 mg-140 mg, or between about 100 mg-130 mg. Preferably, the dose of molecule is administered to the subject is about 0.25 mg, about 24 mg or about 120 mg. A skilled person will understand how to calculate equivalent doses for different BSAs.

In an embodiment, the immune cells are T cells, NK cells, dendritic cells, myeloid cells, macrophages, stem cells or a combination thereof.

In an embodiment, the T cells are CD3+ T cells. In an embodiment, the T cells are cytotoxic T cells, gamma delta T cells, T regulatory cells or iNKT cells.

In an embodiment, the methods described herein provide for an enrichment of CD4+ and/or CD8+ T cells. In another embodiment, at least about 10% of the immune cells are CD8+ cells. In an embodiment, at least about 20% of the immune cells are CD8+ cells. In an embodiment, at least about 30% of the immune cells are CD8+ cells. In an embodiment, at least about 40% of the immune cells are CD8+ cells. In an embodiment, at least about 50% of the immune cells are CD8+ cells. In an embodiment, at least about 60% of the immune cells are CD8+ cells. In an embodiment, between about 10% and about 60% of the immune cells are CD8+ cells. In an embodiment, between about 10% and about 50% of the immune cells are CD8+ cells. In an embodiment, between about 10% and about 40% of the immune cells are CD8+ cells. In an embodiment, between about 10% and about 30% of the immune cells are CD8+ cells.

In another embodiment, the methods described herein provides for an enrichment of CD45RO+CD45RA-T effector memory cells. In another embodiment, the methods described herein comprise an enrichment of CD45RA+CD45RO-T central memory cells.

In an embodiment, where the invention provides for administering immune cells comprising a universal immune receptor covalently bound to the molecule, the method provides for increased CD8+ universal immune receptor cells in the spleen and/or tumour.

In an embodiment, the cells are autologous cells. In an alternate embodiment, the cells are allogeneic.

In an embodiment, the disease is cancer, an infection or an inflammatory disease. Examples of cancers that can be treated using the invention include, but are not limited to, renal cell carcinoma, pancreatic carcinoma, head and neck cancer, prostate cancer, glioblastoma, malignant gliomas, osteosarcoma, colorectal cancer, gastric cancer, malignant mesothelioma, multiple myeloma, ovarian cancer, small cell lung cancer, non-small cell lung cancer, synovial sarcoma, thyroid cancer, breast cancer, melanoma, leukaemia, acute myeloid leukaemia (AML) or lymphoma.

In an embodiment, the subject is a mammal. In an embodiment, the subject is a human.

The present invention further provides for the use of immune cells comprising a universal immune receptor for the manufacture of a medicament for treating a disease in a subject that would benefit from an immune cell therapy, wherein the universal immune receptor may or may not be covalently bound to a molecule which comprises a domain which binds an antigen associated with the disease, wherein the molecule will be administered to the subject at least twice within seven days following administration of the cells, wherein at least 21 days following the seven days the subject will be analysed for responsiveness to the treatment, and wherein the treatment is repeated if the subject has been responsive to the treatment but the disease is still detectable.

Also provided are immune cells comprising a universal immune receptor for use in treating a disease in a subject that would benefit from an immune cell therapy, wherein the universal immune receptor may or may not be covalently bound to a molecule which comprises a domain which binds an antigen associated with the disease, wherein the molecule will be administered to the subject at least twice within seven days following administration of the cells, wherein at least 21 days following the seven days the subject will be analysed for responsiveness to the treatment, and wherein the treatment is repeated if the subject has been responsive to the treatment but the disease is still detectable.

Also provided is the use of immune cells comprising a universal immune receptor for the manufacture of a medicament for stimulating a universal immune receptor mediated immune response to a tumour in a subject, wherein the universal immune receptor may or may not be covalently bound to a molecule which comprises a domain which binds an antigen associated with the tumour, wherein the molecule will be administered to the subject at least twice within seven days following administration of the cells, wherein at least 21 days following the seven days the subject will be analysed for responsiveness to the treatment, and wherein the treatment is repeated if the subject has been responsive to the treatment but the tumour is still detectable.

Also provided are immune cells comprising a universal immune receptor for use in stimulating a universal immune receptor mediated immune response to a tumour in a subject, wherein the universal immune receptor may or may not be covalently bound to a molecule which comprises a domain which binds an antigen associated with the tumour, wherein the molecule will be administered to the subject at least twice within seven days following administration of the cells, wherein at least 21 days following the seven days the subject will be analysed for responsiveness to the treatment, and wherein the treatment is repeated if the subject has been responsive to the treatment but the tumour is still detectable.

Also provided is the use of immune cells comprising a universal immune receptor for the manufacture of a medicament for treating a disease in a subject that would benefit from an immune cell therapy, wherein the universal immune receptor may or may not be covalently bound to a molecule which comprises a domain which binds an antigen associated with the disease, wherein the molecule will be administered to the subject every two or three days for between 14 days and 28 days following administration of the cells, and wherein the treatment is repeated if the subject has been responsive to the treatment but the disease is still detectable.

Also provided are immune cells comprising a universal immune receptor for use in treating a disease in a subject that would benefit from an immune cell therapy, wherein the universal immune receptor may or may not be covalently bound to a molecule which comprises a domain which binds an antigen associated with the disease, wherein the molecule will be administered to the subject every two or three days for between 14 days and 28 days following administration of the cells, and wherein the treatment is repeated if the subject has been responsive to the treatment but the disease is still detectable.

Also provided is the use of immune cells comprising a universal immune receptor for the manufacture of a medicament for stimulating a universal immune receptor mediated immune response to a tumour in a subject, wherein the universal immune receptor may or may not be covalently bound to a molecule which comprises a domain which binds an antigen associated with the tumour, wherein the molecule will be administered to the subject every two or three days for between 14 days and 28 days following administration of the cells, and wherein the treatment is repeated if the subject has been responsive to the treatment but the tumour is still detectable.

Also provided are immune cells comprising a universal immune receptor for use in stimulating a universal immune receptor mediated immune response to a tumour in a subject, wherein the universal immune receptor may or may not be covalently bound to a molecule which comprises a domain which binds an antigen associated with the tumour, wherein the molecule will be administered to the subject every two or three days for between 14 days and 28 days following administration of the cells, and wherein the treatment is repeated if the subject has been responsive to the treatment but the tumour is still detectable.

In another aspect, the present invention provides a substantially purified and/or recombinant polypeptide comprising a sequence of amino acids provided as SEQ ID NO: 5 or SEQ ID NO:6, or a sequence of amino acids at least 90% identical to one or both of SEQ ID NO:5 and SEQ ID NO:6, wherein the polypeptide is capable of covalently binding to a protein comprising SpyCatcher and binding a HER2 receptor on a cancer cell.

In another aspect, the present invention provides a substantially purified and/or recombinant polypeptide comprising a sequence of amino acids provided as SEQ ID NO: 7 and/or SEQ ID NO: 10, or provided as SEQ ID NO:8 and/or SEQ ID NO:9 or having a sequence of amino acids at least 90% identical thereto, wherein the polypeptide is capable of covalently binding to a protein comprising SpyCatcher and binding an EGFRvIII receptor on a cancer cell.

In another aspect, the present invention provides a substantially purified and/or recombinant polypeptide comprising a sequence of amino acids provided as SEQ ID NO: 11 and/or SEQ ID NO: 14, or provided as SEQ ID NO: 12 and/or SEQ ID NO: 13 or having a sequence of amino acids at least 90% identical thereto, wherein the polypeptide is capable of covalently binding to a protein comprising SpyCatcher and binding an IL-13Ra2 receptor on a cancer cell.

In another aspect, the present invention provides a substantially purified and/or recombinant polypeptide comprising a sequence of amino acids provided as SEQ ID NO: 15 and/or SEQ ID NO: 16, or a sequence of amino acids at least 90% identical thereto, wherein the polypeptide is capable of covalently binding to a protein comprising Spy Catcher and binding an CD33 receptor on a cancer cell.

In another aspect, the present invention provides a substantially purified and/or recombinant polypeptide comprising a sequence of amino acids provided as SEQ ID NO: 17 and/or SEQ ID NO: 18, or a sequence of amino acids at least 90% identical thereto, wherein the polypeptide is capable of covalently binding to a protein comprising Spy Catcher and binding an C-type lectin-like (CLL1) receptor on a cancer cell.

In another aspect, the present invention provides an isolated and/or exogenous polynucleotide encoding the polypeptide of the invention.

In a further aspect the present invention provides a vector comprising the polynucleotide of the invention.

In yet another aspect, the present invention provides an isolated transgenic cell comprising a polynucleotide of the invention and/or a vector of the invention. In an embodiment, the cell is a bacterial cell or a mammalian cell.

In yet another aspect, the present invention provides a pharmaceutical composition comprising immune cells comprising a universal immune receptor which may or may not be covalently bound to a molecule which comprises a domain which binds an antigen associated with a disease. In an embodiment, the domain comprises one or more or all of SEQ ID NO:5, SEQ ID NO:6, SEQ ID NO: 7, SEQ ID NO:8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, or a sequence of amino acids at least 90% identical thereto.

In another aspect the present invention provides a method of producing a polypeptide of the invention, the method comprising culturing cells of the invention, and purifying the polypeptide from the cells or culture medium.

In an embodiment, the cells are cultured in the presence of an immune cell activator, preferably IL-2, an anti-CD3 antibody or an anti-CD28 antibody. In another embodiment, the immune cell activator increases expression of Tim-3 and/or PD-1. In yet another embodiment, at least about 30% of the immune cells are Tim-3 and/or PD-1 positive.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIGS. 1 to 5—Examples of dosing schedules.

FIG. 6—Dosing regimen of binders regulates functional UIR expression in vivo. (A) Expression of ‘unarmed’ OmniCAR T cells (FLAG+ only), and ‘armed’ OmniCAR T cells (FLAG+IgG+). (B) ‘Armed’ OmniCAR receptor detected by IgG MFI in CD8+ FLAG+ or CD4+ FLAG+ CAR T cells armed with increasing concentrations of antibody binders. (C) Production of cytokines IFNγ, TNF and IL-2 by OmniCAR T cells armed with increasing concentrations of binders co-cultured for 24 hours with MDA-MB231-HER2 tumours. (D) FACS plots showing % armed OmniCAR receptors on T cells isolated from blood day 1 or day 7 after adoptive transfer. (E) % IgG+ of CD8+ FLAG+CAR T cells from blood at day 1 post transfer for groups dosed with varying doses of binders every 3-4 days. (F) Counts of armed IgG+ FLAG+ OmniCAR T cells in blood day I post transfer for groups dosed with varying doses of binders every 3-4 days.

FIG. 7—Dosing regimen of binders regulates T cell memory phenotype, expansion, and persistence in vivo. (A) Schematic diagram of treatment regimens, low dose=1 ug, high dose=25 ug. Pre-conditioned tumour bearing mice were treated with a single dose of 10-20 million unarmed or pre-armed OmniCAR T cells and further dosed with antibody binders according to treatment plan 1-3. (B) Counts of CD8+ FLAG+ cells/uL of blood at day 1 post transfer. (C) MFI of IgG staining on OmniCAR T cells from blood on day 7 post transfer. (D) Counts of total CD8+ T cells/uL of blood at day 7 post transfer for non-transduced vs unarmed OmniCAR groups. (E) Memory phenotype of CD45RO+CD45RA− (T effector memory) or CD45RA+CD45RO− (T central memory) populations. Data shown as mean±SEM.

FIG. 8—Dosing regimen of binders modulates anti-tumour efficacy in vivo. (A) Tumour growth curves for mice treated with pre-armed OmniCAR T cells and dosed with high dose of binders. (B) Spleens or (C) Tumours were extracted at endpoint of therapy and CD8+FLAG+ OmniCAR T cells were counted. (D) % TIM3+PD1+ population of CD8+FLAG+ OmniCAR TILs from tumours at endpoint. (E) Bioluminescence imaging to determine tumour burden over time in a mouse model of acute myeloid leukemia (AML). NSG mice were give 5 million KG-1 cells and the animals were either left untreated (control) or they were given pre-armed OmniCAR-T cells and 25 ug of CD33 and CLL-1 binder on days 3, 6 and 9 post CAR-T transfer. Data shown as mean±SEM.

FIG. 9—Dosing regimen and specific design of binders regulates OmniCAR antigen-independent signaling or antigen-dependent signalling. (A) TIM3 expression of unstimulated or stimulated (OKT3) T cells after 72 hours subset by % of CD8+FLAG+ (Top) or % of CD4+FLAG+ (Down). (B) PD-1 expression (Left) of unstimulated or stimulated (OKT3) T cells after 72 hours subset by % of CD8+FLAG+ (Top) or % of CD4+FLAG+ (Down). *p<0.05, **p<0.01, ***p<0.001, ****p<0.0001, data shown as mean±SEM.

FIG. 10—Metronomic dosing to target multiple tumour antigens sequentially or simultaneously. (A) Counts of mixed tumour culture of U251MG-HER2 (GFP) and U251MG-EGFRviii (mCherry) tumours. (B) anti-HER2 armed OmniCAR T cells cocultured with mixed tumour culture. (C) anti-EGFRviii armed OmniCAR T cells cocultured with mixed tumour culture. (D) anti-EGFRviii armed OmniCAR T cells cocultured with mixed tumour culture with (HER2LT switching) or without (no switching) addition of anti-HER2 binders at 20 hours post coculture. Data shown as mean±SD. (E) (Top) Histogram of expression of three binders on the same OmniCAR T cell sample (Bottom). FACs plots of binder expression in each dual combination on the same OmniCAR T cell product. (F) Determination of the presence of HER2 and EGFRVIII antibody binders in sera of mice.

FIG. 11—Model of antigen-independent tonic signalling in OmniCAR vs conventional CAR T cells to regulate memory and anti-tumour functional capacity. (A) Schematic diagram of metronomic dosing encompassing temporal, dose modulation or multi-binder combination strategies. (B) Model of inter-relation between antigen-independent modulation of tonic signalling, memory phenotype, and functional/anti-tumour capacity.

### KEY TO THE SEQUENCE LISTING

SEQ ID NO:1—SpyCatcher universal immune receptor amino acid sequence
SEQ ID NO:2—Nucleotide sequence encoding Spy Catcher universal immune receptor
SEQ ID NO:3—Standard Her2-Spy Tag binder with N-terminal signal
SEQ ID NO:4—Short half-life Her2-Spy Tag binder with N-terminal signal
SEQ ID NO:5—Standard Her2-Spy Tag binder without N-terminal signal
SEQ ID NO:6—Short half-life Her2-SpyTag binder without N-terminal signal
SEQ ID NO:7—EGFRvIII heavy chain amino acid sequence
SEQ ID NO:8—EGFRvIII heavy chain with SpyTag amino acid sequence
SEQ ID NO: 9—EGFRvIII light chain amino acid sequence
SEQ ID NO:10—EGFRvIII light chain with Spy Tag amino acid sequence
SEQ ID NO:11—IL-13Ra2 heavy chain amino acid sequence
SEQ ID NO: 12—IL-13Ra2 heavy chain with Spy Tag amino acid sequence
SEQ ID NO: 13—IL-13Ra2 light chain amino acid sequence
SEQ ID NO:14—IL-13Ra2 light chain with Spy Tag amino acid sequence
SEQ ID NO:15—CD33 heavy chain amino acid sequence
SEQ ID NO:16—CD33 light chain amino acid sequence with SpyTag amino acid
sequence SEQ ID NO:17—CLL1 heavy chain amino acid sequence
SEQ ID NO:18—CLL1 light chain amino acid sequence with SpyTag amino acid sequence

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, cell based immunotherapy, molecular genetics, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, more preferably +/−5%, more preferably +/−1%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “subject” can be any animal. In one embodiment, the animal is a vertebrate. For example, the animal can be a mammal, avian, chordate, amphibian or reptile. Exemplary subjects include but are not limited to human, primate, livestock (e.g. sheep, cow, chicken, horse, donkey, pig), companion animals (e.g. dogs, cats), laboratory test animals (e.g. mice, rabbits, rats, guinea pigs, hamsters), captive wild animal (e.g. fox, deer). In one embodiment, the mammal is a human. In an embodiment, a method of the invention is for veterinary use.

The terms “treating” or “treatment” as used herein, refer to both direct treatment of a subject by a medical professional (e.g., by administering a therapeutic agent to the subject), or indirect treatment, effected, by at least one party, (e.g., a medical doctor, a nurse, a pharmacist, or a pharmaceutical sales representative) by providing instructions, in any form, that (i) instruct a subject to self-treat according to a claimed method (e.g., self-administer a drug) or (ii) instruct a third party to treat a subject according to a claimed method. Also encompassed within the meaning of the term “treating” or “treatment” are prevention or reduction of the disease to be treated, e.g., by administering a therapeutic at a sufficiently early phase of disease to prevent or slow its progression.

As used herein, the term “the subject has been responsive to the treatment but the disease is still detectable” refers to a detectable reduction (such as at least a 75% reduction, at least a 50% reduction or at least a 25% reduction) in the disease (such as a reduction in tumour load) but the disease is still present. Methods for detecting diseases which can be treated using the methods of the invention are well known in the art and include imaging (such as PET, PET_SPECT and MRI), cell detection and pathogen detection techniques.

As used herein, “cytokine release syndrome” (CRS) refers to is an acute systemic inflammatory syndrome characterized by fever and multiple organ dysfunction that is associated with chimeric antigen receptor cell therapy, therapeutic antibodies, and haploidentical allogeneic transplantation.

A polypeptide may be defined by the extent of identity (% identity) of its amino acid sequence to a reference amino acid sequence, or by having a greater % identity to one reference amino acid sequence than to another. The % identity of a polypeptide to a reference amino acid sequence is typically determined by GAP analysis (Needleman and Wunsch, 1970: GCG program) with parameters of a gap creation penalty=5, and a gap extension penalty=0.3. The query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns two sequences over the entire length of the reference amino acid sequence.

With regard to a defined polypeptide, it will be appreciated that % identity figures higher than those provided herein will encompass preferred embodiments. Thus, where applicable, in light of the minimum % identity figures, it is preferred that the polypeptide comprises an amino acid sequence which is at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO. In an embodiment, for each of the ranges listed above, the % identity does not include 100% i.e. the amino acid sequence is different to the nominated SEQ ID NO.

The terms “combination therapy”, “administered in combination” or “co-administration” or the like, as used herein, are meant to encompass administration of the selected therapeutic agents to a single subject, and are intended to include treatment regimens in which the agents are administered by the same or different route of administration or at the same or different time.

### Universal Immune Receptors

As used herein, a “universal immune receptor” or “UIR” is a chimeric antigen receptor system where the immune cell recombinantly expresses a protein comprising an extracellular binding domain bound to an extracellular hinge region, which is in turn bound to a transmembrane domain which is in turn bound to an immune cell receptor intracellular signaling domain. The system further comprises a soluble molecule which comprises a first domain that binds the extracellular binding domain and a second domain which binds an antigen associated with a disease (such as a cancer antigen on the surface of a cancer cell). As used herein, the term “universal immune receptor” can refer to the molecule bound (also referred to as armed) or not bound (also referred to an unarmed) to the extracellular binding domain. UIRs for use in the invention form a covalent bond when the first domain binds the extracellular binding domain.

An example of a universal immune receptor for use in the invention is the Spy Tag/Spy Catcher system (WO 2017/112784). The term “Spy Tag/SpyCatcher system” encompasses each version of the system such as version 1 (U.S. Pat. No. 9,547,003), version 2 (WO 2018/197854) and version 3 (WO 2020/183198). As another example, a universal immune receptor for use in the invention is the SnoopTag/SnoopCatcher system (Veggiani et al., 2016: WO 2016/193746).

The term “chimeric antigen receptor” or alternatively “CAR” in the context of the invention refers to a polypeptide, which when in an immune cell, provides the cell with specificity for a target cell once the molecule is covalently bound to the extracellular binding domain bound, for example a cancer cell, and with intracellular signal generation.

CARs (UIRs) can be used to generate immune cells, such as T cells, dendritic cells, or natural killer (NK) cells, specific for selected targets. Suitable constructs for generating CARs are described in U.S. Pat. Nos. 5,843,728; 5,851,828; 5,912,170; 6,004,811; 6,284,240; 6,392,013; 6,410,014; 6,753,162; 8,211,422; and WO9215322. Alternative CAR constructs can be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signalling domains of either CD3C or FcRy or scFv-FcRy (see, e.g., U.S. Pat. Nos. 7,741,465; 5,912,172; and U.S. Pat. No. 5,906,936). Second-generation CARs incorporate the intracellular domains of one or more costimulatory molecules, such as CD28, CD28z, OX40 (CD134), or 4-1BB (CD137) within the endodomain, e.g., scFv-CD28/OX40/4 BB-CD3 (see, e.g., U.S. Pat. Nos. 8,911,993; 8,916,381; 8,975,071; 9,101,584; 9,102,760; 9,102,761). Third-generation CARs include a combination of costimulatory endodomains, such a CD3C-chain, CD97, GDI 1a-CD18, CD2, ICOS, CD27, CD154, CDS, OX40, 4-1BB, or CD28 signalling domains, e.g., scFv-CD28-4 BB-CD3C or scFv-CD28-OX40-CD3Q (see, e.g., U.S. Pat. Nos. 8,906,682; 8,399,645; 5,686,281; WO2014134165; and WO2012079000). In some embodiments, costimulation can be coordinated by expressing CARs in antigen-specific T cells, chosen so as to be activated and expanded following, for example, interaction with antigen on professional antigen-presenting cells, with costimulation. Additional engineered receptors can be provided on the immune cells, e.g., to improve targeting of a T-cell attack and/or minimize side effects.

The skilled artisan will be aware that an “antibody” is generally considered to be a protein that comprises at least one variable region made up of one or more polypeptide chains, e.g., a polypeptide comprising a V_{L} and/or a polypeptide comprising a V_{H}. An antibody also generally comprises constant domains, some of which can be arranged into a constant region, which includes a constant fragment or fragment crystallizable (Fc) region, in the case of a heavy chain. A V_{H} and a V_{L} interact to form a Fv comprising an antigen binding site that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies and chimeric antibodies.

The terms “full-length antibody.” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

The term “antibody fragment” as used herein includes antibody fragments which retain the capability of binding to a target antigen, for example, Fab, Fab′, F(ab′) 2, Fv, scFv fragments, other antigen-binding subsequences of antibodies and can include those produced by the modification of whole antibodies or those synthesized de novo using recombinant DNA technologies, and the corresponding fragments obtained from antibodies other than IgG. These antibody fragments are obtained using conventional procedures, such as proteolytic fragmentation procedures, as described in J. Goding, Monoclonal Antibodies: Principles and Practice, pp 98-118 (N.Y. Academic Press 1983), as well as by other techniques known to those with skill in the art. The fragments are screened for utility in the same manner as are intact antibodies.

Suitable antibodies or antigen binding fragments include, but are not limited to, IgG, IgA, IgM, IgE, monoclonal antibody, Fab′, rIgG (half antibody), f (ab) 2, nanobody, chimeric antibody, scFv, scFv multimer, single domain antibody or single domain fusion antibody. In some embodiments, the antibody or antibody-like molecule is a monoclonal antibody or an antigen binding fragments thereof. The term “monoclonal antibody,” as used herein, refers to a preparation of antibody molecules of single molecular composition. A monoclonal antibody displays a single binding specificity and affinity for a particular epitope.

In an embodiment, the molecule is a polypeptide. In an embodiment, the molecule is a single polypeptide chain encoded by a single open reading frame.

In an embodiment, the molecule further comprises a third domain which is a labelling agent. In some embodiments, the labelling agent is selected from the group consisting of myc-tag. FLAG-tag. His-tag. HA-tag, a fluorescent protein (e.g. green fluorescent protein (GFP)), a fluorophore (e.g. tetramethylrhodamine (TRITC), fluorescein isothiocyanate (FITC)), dinitrophenol, peridinin chlorophyll protein complex, green fluorescent protein, phycoerythrin (PE), histidine, biotin, streptavidin, avidin, horse radish peroxidase, palmitoylation, nitrosylation, alkalanine phosphatase, glucose oxidase, Glutathione S-transferase (GST), maltose binding protein, a radioisotope, and any types of compounds used for radioisotope labeling including, 1,4,7,10-tetraazacyclododecane-1,4,7, 10-tetraacetic acid (DOTA), di ethylene triamine pentaacetic acid (DTP A), and 1,4,7-triazacyclononane-1,4,7-triacetic acid (NOTA).

The molecule comprising a domain which binds an antigen associated with the disease can be produced by any means known in the art. In one embodiment, the molecule is produced and purified from a recombinant cell expressing the molecule. In another embodiment, the molecule is synthesised.

### Methods of Preparing UIR-Expressing Cells

### Sources of Cells

Prior to expansion, and possible genetic modification or other modification, a cell population comprising or consisting of immune cells such as T cells, dendritic cells, macrophages, natural killer (NK) cells or a combination thereof, can be obtained from a subject. Immune cells can be obtained from a number of sources, including peripheral blood mononuclear cells, bone marrow, lymph node tissue, cord blood, thymus tissue, tissue from a site of infection, ascites, pleural effusion, spleen tissue, and tumours.

In certain embodiments of the present disclosure, immune cells, e.g., T cells, can be obtained from a unit of blood collected from a subject using any number of techniques known to the skilled artisan, such as Ficoll™ separation. In one preferred embodiment, cells from the circulating blood of an individual are obtained by apheresis. The apheresis product typically contains lymphocytes, including T cells, monocytes, granulocytes, B cells, dendritic cells, other nucleated white blood cells, red blood cells, and platelets. In one embodiment, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al. (2015).

In one embodiment, T cells are isolated from peripheral blood lymphocytes by lysing the red blood cells and depleting the monocytes, for example, by centrifugation through a PERCOLL™ gradient or by counterflow centrifugal elutriation.

The methods described herein can include, e.g., selection of a specific subpopulation of immune cells, e.g., T cells, that are a T regulatory cell-depleted population. A CD25+ depleted cell population, for example, can be obtained using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In one embodiment, T regulatory (T_{REG}) cells, e.g., CD25+ T cells, are removed from the population using an anti-CD25 antibody, or fragment thereof, or a CD25-binding ligand, IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

Without wishing to be bound by a particular theory, decreasing the level of negative regulators of immune cells (e.g., decreasing the number of unwanted immune cells, e.g., T_{REG} cells), in a subject prior to apheresis or during manufacturing of a UIR-expressing cell product can reduce the risk of subject relapse. For example, methods of depleting T_{REG} cells are known in the art. Methods of decreasing T_{REG} cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) T_{REG} cells prior to manufacturing of the UIR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the UIR-expressing cell (e.g., T cell, NK cell) product.

Cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune cell therapy for any number of diseases or conditions that would benefit from immune cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, Cytoxan, fludarabine, cyclosporin, FK506, rapamycin, mycophenolic acid, steroids, FR901228, and irradiation.

### Methods of Making UIR-Expressing Cells

In an embodiment, a method of the invention includes the making of UIR-expressing cells by introducing a vector or nucleic acid encoding a UIR into a cell. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, for example, Sambrook Molecular Cloning: A Laboratory Manual, volumes 1-4, Cold Spring Harbor Press). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (see, for example, U.S. Pat. Nos. 5,350,674 and 5,585,362).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

An exemplary non-viral delivery vehicle is a liposome. The use of lipid formulations is contemplated for the introduction of the nucleic acids into a host cell (in vitro, ex vivo or in vivo). In another embodiment, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma Aldrich: dicetyl phosphate (“DCP”) can be obtained from K & K Laboratories; cholesterol (“Choi”) can be obtained from Calbiochem-Behring: dimyristyl phosphatidylglycerol (“DMPG”) and other lipids may be obtained from Avanti Polar Lipids, Inc. for example. Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium.

Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR: “biochemical” assays, such as detecting the presence or absence of a particular peptide, e.g., by immunological means (ELISAs and Western blots) or by assays described herein to identify agents falling within the scope of the invention.

### Methods of Culturing and Expanding Immune Cells

Immune cells such as T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and US20060121005.

Expanding the T cells by the methods disclosed herein can multiply the cells by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers there between. In one embodiment, the T cells expand in the range of about 20 fold to about 50 fold.

In an embodiment, the cells are cultured for between about 7 days and about 14 days, or about 7 days to about 10 days.

Generally, a population of immune cells e.g., T regulatory cell depleted cells, may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For costimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., 1998; Haanen et al., 1999; Garland et al., 1999).

Conditions appropriate for immune cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGF, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

A procedure for ex vivo expansion of hematopoietic stem and progenitor cells is described in U.S. Pat. No. 5,199,942, can be applied to the cells of the present invention. Other suitable methods are known in the art, therefore the present invention is not limited to any particular method of ex vivo expansion of the cells. Briefly, ex vivo culture and expansion of immune cells (e.g., T cells) comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

### Immune Cells

As used herein, the phrase “immune cell” refers to a cell which is capable of affecting or inducing an immune response upon recognition of an antigen. In some embodiments, the immune cell is a T cell, a natural killer (NK) cell, a macrophage, a dendritic cell or a stem cell. In some embodiments, the cell is a mammalian cell. In some embodiments, the cell is a human cell. The cells may be autologous or allogeneic to the subject to which they are administered.

Examples of stem cells useful for the invention include, but are not limited to, haematopoietic stem/progenitor cells and induced pluripotent stem cells.

As used herein, the phrase “cytotoxicity activity” refers to the ability of an immune cell, such as an NK cell, to destroy living cells.

As used herein, the term “immune response” has its ordinary meaning in the art, and includes both humoral and cellular immunity. An immune response can manifest as one or more of, the development of anti-antigen antibodies, expansion of antigen-specific T cells, increase in tumour infiltrating-lymphocytes (TILs), development of an anti-tumour or anti-tumour antigen delayed-type hypersensitivity (DTH) response, clearance of the pathogen, suppression of pathogen and/or tumour growth and/or spread, tumour reduction, reduction or elimination of metastases, increased time to relapse, increased time of pathogen or tumour free survival, and increased time of survival. An immune response may be mediated by one or more of, B-cell activation, T-cell activation, natural killer cell activation, activation of antigen presenting cells (e.g., B cells, DCs, monocytes and/or macrophages), cytokine production, chemokine production, specific cell surface marker expression, in particular, expression of co-stimulatory molecules. The immune response may be characterized by a humoral, cellular, Th1 or Th2 response, or combinations thereof. In an embodiment, the immune response is an innate immune response.

### T Cells

In some embodiments, the immune cell is a T cell e.g. a UIR-T cell. T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are several subsets of T cells, each with a distinct function.

In an embodiment, the T cells are or include central memory (TCM) T cells. TCM cells patrol lymph nodes, providing central immunosurveillance against known pathogens, but have not been described as conducting primary tissue immunosurveillance. In an embodiment, TCM cells produced using a method of the invention include CD45RO+ CD62L+ T cells, preferably CD45RO+CD62L^{hi} T cells. Such cells may also be CCR7+.

In an embodiment, the T cells are or include central memory stem cell (T_{SCM}) T cells. T_{SCM} cells a rare subset of memory lymphocytes endowed with the stem cell-like ability to self-renew and the multipotent capacity to reconstitute the entire spectrum of memory and effector subset. In an embodiment, the T_{SCM} cells include CD27⁺CD95⁺ T cells.

As used herein, a regulatory T cell (T_{REG}), or variations thereof, refers to a population of T cells which are crucial for the maintenance of immunological tolerance. Their major role is to shut down T cell-mediated immunity toward the end of an immune reaction and to suppress auto-reactive T cells that escaped the process of negative selection in the thymus. Two major classes of CD4+ T_{REG} cells have been described-Foxp3+ and Foxp3−.

Gamma delta (γδ) T cells are the prototype of ‘unconventional’ T cells and represent a relatively small subset of T cells in peripheral blood. They are defined by expression of heterodimeric T-cell receptors (TCRs) composed of γ and δ chains. This sets them apart from CD4+ helper T cells and CD8+ cytotoxic T cells that express αβ TCRs.

iNKT (invariant NKT) cells are classified as innate-like lymphocytes that promptly secrete Th1 or Th2 cytokines when antigens bind with TCRs. Activated INKT cells can regulate the adaptive immune response via the recruitment, activation, or modulation of the responses of NK cells, DCs, B cells, and T cells.

In an embodiment, the T cells are naïve T cells. As used herein, the term “naïve T cells” refers to a population of T cells that has matured and been released by the thymus but has not yet encountered its corresponding antigen. In other words, naïve T cells are in the stage between maturity and activation. Naïve T cells are commonly characterized by the surface expression of L-selectin (CD62L) and C—C Chemokine receptor type 7 (CCR7): the absence of the activation markers CD25, CD44 or CD69; and the absence of memory CD45RO isoform. They also express functional IL-7 receptors, consisting of subunits IL-7 receptor-α, CD127, and common-γ chain, CD132.

A T cell lacking a functional endogenous T cell receptor (TCR) can be, e.g., engineered such that it does not express any functional TCR on its surface, engineered such that it does not express one or more subunits that comprise a functional TCR or engineered such that it produces very little functional TCR on its surface. Alternatively, the T cell can express a substantially impaired TCR, e.g., by expression of mutated or truncated forms of one or more of the subunits of the TCR. The term “substantially impaired TCR” means that this TCR will not elicit an adverse immune reaction in a host.

A T cell described herein can be, e.g., engineered such that it does not express a functional HLA on its surface. For example, a T cell described herein, can be engineered such that cell surface expression HLA, e.g., HLA class 1 and/or HLA class II, is downregulated. In some embodiments, the T cell can lack a functional TCR and a functional HLA, e.g., HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

### Natural Killer Cells

In some embodiments, the immune cell is a natural killer cell. Natural-killer (NK) cells are CD56 CD3 large granular lymphocytes that can kill infected and transformed cells, and constitute a critical cellular subset of the innate immune system. Unlike cytotoxic CD8+ T lymphocytes, NK cells launch cytotoxicity against tumour cells without the requirement for prior sensitization, and can also eradicate MHC-I−negative cells. In an embodiment, the NK cells are CD3−CD56+CD7+CD127− NKp46+ T-bet+Eomes+. In an embodiment, cytotoxic NK cells CD56^{dim} CD16+.

### Dendritic Cells

In some embodiments, the immune cell is a dendritic cell. Dendritic cells are a heterogeneous group of specialized antigen-presenting cells that originate in the bone marrow from CD34+ stem cells and express major histocompatibility complex (MHC) class II molecules. Mature dendritic cells are able to prime, activate and expand effector immune cells, such as T cells and NK cells. Dendritic cell therapy is known in the art (see, e.g. Sabado et al., 2017). Briefly, dendritic cells can be isolated from a patient, exposed to a disease-specific antigen, for example a cancer specific antigen, or genetically modified to express a UIR, or a disease specific antigen, and are then infused back into the patient where they prime, activate and expand effector immune cells, for example T cells.

### Myeloid Cells

In some embodiments, the immune cell is a myeloid cell. Granulocytes, monocytes, macrophages, and dendritic cells represent a subgroup of leukocytes, collectively called myeloid cells. They circulate through the blood and lymphatic system and are rapidly recruited to sites of tissue damage and infection via various chemokine receptors. Within the tissues they are activated for phagocytosis as well as secretion of inflammatory cytokines, thereby playing major roles in protective immunity. Myeloid cell therapies are known in the art and may be useful in the treatment of cancer, infection or disease. For instance, myeloid cells are known to be abundant in the tumour stroma and the presence of these cells may influence patient outcome in many cancer types. Briefly, myeloid cells can be isolated from a patient, exposed to a disease-specific antigen, for example a cancer specific antigen, or genetically modified to express a UIR, or a disease specific antigen, and are then infused back into the patient where they prime, activate and expand effector immune cells, for example T cells.

### Macrophages

Macrophages are specialised cells involved in the detection, phagocytosis and destruction of bacteria and other harmful organisms. In addition, they can also present antigens to T cells and initiate inflammation by releasing molecules (known as cytokines) that activate other cells.

### UIR-Expressing Cell Therapy

UIR cell therapy is a type of cellular therapy where immune cells (e.g., T cells) are genetically modified to express a UIR and the UIR-expressing cell (e.g. a UIR-T cell) is infused to a recipient in need thereof. The infused cell is able to kill diseased cells expressing the target of the UIR in the recipient. Unlike antibody therapies, UIR-modified immune cells (e.g., UIR-T cells) are able to replicate in vivo resulting in long-term persistence that can lead to sustained tumour control. In various embodiments, the UIR cells are administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the UIR-cell to the patient.

In an embodiment, when administered to the subject the UIR cells are pre-armed. More specifically, the cells have been exposed to the molecule under conditions such that it covalently binds the UIR prior to being administered to the subject, and hence are capable of binding a target cell (such as a cancer cell) when they are administered.

In an embodiment, when administered to the subject the UIR cells are not pre-armed. More specifically, the cells have not been exposed to the molecule under conditions such that it covalently binds the UIR prior to being administered to the subject. In this embodiment, for the cells to be functional the molecule is also administered to the subject for the cells become armed in vivo.

In one embodiment, pre-armed UIR cells are administered followed by administration of the molecule every 3 days, such as days 1, 4 and 6, for about 21 days.

In an embodiment, pre-armed UIR cells are administered followed by administration of the molecule twice a week, such as days 3 and 6, followed by about 21 days of rest.

In an embodiment, unarmed UIR cells are administered followed by administration of the molecule twice a week, such as days 3 and 6, followed by about 21 days of rest.

In an embodiment, pre-armed UIR cells are administered followed by administration of the molecule thrice a week, such as days 1, 4 and 6, followed by about 21 days of rest.

In an embodiment, unarmed UIR cells are administered followed by administration of the molecule thrice a week, such as days 1, 4 and 6, followed by about 49 days of rest.

The invention may be conducted in a number of cycles. For instance, following the first cycle as defined herein, the subject is analysed to determine if they have been responsive to the therapy. If the subject is responsive to the treatment but the disease is still detectable the subject may be subjected to a second cycle of the therapy. Furthermore, if, following the second cycle the subject is responsive to the treatment but the disease is still detectable the subject may be subjected to a third cycle of the therapy, and so on.

In an embodiment, the subject is rested before determining if they have been responsive to the therapy. In an embodiment, the subject is rested for about 21 days. In an embodiment, the subject is rested for about 28 days. In an embodiment, the subject is rested for about 35 days. In an embodiment, the subject is rested for about 42 days. In an embodiment, the subject is rested for about 49 days.

The invention also includes a type of cellular therapy where immune cells (e.g., T cells) are modified, e.g., by in vitro transcribed RNA, to transiently express a UIR and the UIR-T cell is infused to a recipient in need thereof. The infused cell is able to kill tumour cells in the recipient. Thus, in various embodiments, the immune cells (e.g., UIR-T cells) administered to the patient, is present for less than one month, e.g., three weeks, two weeks, one week, after administration of the UIR-T cell to the patient. Without wishing to be bound by any particular theory, the anti-tumour immunity response elicited by the UIR-T cells may be an active or a passive immune response, or alternatively may be due to a direct vs indirect immune response.

Where the invention contemplates methods for stimulating a universal immune receptor mediated immune response to a tumour in a subject, it is envisaged that the immune response elicited by the UIR-T cells, whether an active or a passive immune response, or a direct vs indirect immune response, is sufficient to treat the cancer in the subject.

As noted above, ex vivo procedures are well known in the art and are described above. Briefly, cells are isolated from a mammal (e.g., a human) and genetically modified (i.e., transduced or transfected in vitro) with a vector expressing a UIR. The UIR-expressing cell (e.g., a UIR-T cell) can be administered to a mammalian recipient to provide a therapeutic benefit. The mammalian recipient may be a human and the UIR-expressing cell can be autologous with respect to the recipient. Alternatively, the cells can be allogeneic, syngeneic or xenogeneic with respect to the recipient.

The UIR cells of the present invention may be administered either alone, or as a pharmaceutical composition in combination with diluents and/or with other components such as IL-2 or other cytokines or cell populations, as described herein. Immune cells may be administered either alone, or as a pharmaceutical composition in combination with diluents and/or with other components such as IL-2, IL-15, or other cytokines or cell populations. Briefly, pharmaceutical compositions may comprise immune cells as described herein, in combination with one or more pharmaceutically or physiologically acceptable carriers, diluents or excipients. Such compositions may comprise buffers such as neutral buffered saline, phosphate buffered saline and the like: carbohydrates such as glucose, mannose, sucrose or dextrans, mannitol: proteins: polypeptides or amino acids such as glycine: antioxidants: chelating agents such as EDTA or glutathione: adjuvants (e.g., aluminium hydroxide); and preservatives. Compositions for use in the disclosed methods are in some embodiments formulated for intravenous administration.

A pharmaceutical composition comprising the cells described herein may be administered at a dosage of 10⁴ to 10⁹ cells/kg body weight, such as 10⁵ to 10⁶ cells/kg body weight, including all integer values within those ranges. Cell compositions may also be administered multiple times at these dosages. The cells can be administered by using infusion techniques that are commonly known in immunotherapy (see, e.g., Rosenberg et al., 1988). The optimal dosage and treatment regime for a particular patient can readily be determined by one skilled in the art of medicine by monitoring the patient for signs of disease and adjusting the treatment accordingly.

A pharmaceutical composition comprising the molecule (binder) described herein may be administered at a dosage of, for example, between 0.5 mg to 5 mg per kg.

In certain embodiments, it may be desired to administer activated immune cells to a subject and then subsequently re-draw blood (or have an apheresis performed), activate and expand the immune cells therefrom, and reinfuse the patient with these activated and expanded cells. This process can be carried out multiple times every few weeks. In certain embodiments, Immune cells can be activated from blood draws of from 10 cc to 400 cc. In certain embodiments, immune cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of immune cells.

### Combination Therapies

The immune cells, such as UIR-T cells of the present invention, or produced by the methods of the present invention, may be co-formulated with and/or administered in combination with one or more additional therapeutically active component(s) selected from the group consisting of: a PRLR antagonist (e.g., an anti-PRLR antibody or small molecule inhibitor of PRLR), an EGFR antagonist (e.g., an anti-EGFR antibody [e.g., cetuximab or panitumumab] or small molecule inhibitor of EGFR [e.g., gefitinib or erlotinib]), an antagonist of another EGFR family member such as Her2/ErbB2, ErbB3 or ErbB4 (e.g., anti-ErbB2 [e.g., trastuzumab or T-DM1], anti-ErbB3 or anti-ErbB4 antibody or small molecule inhibitor of ErbB2, ErbB3 or ErbB4 activity), a cMET antagonist (e.g., an anti-cMET antibody), an IGF1R antagonist (e.g., an anti-IGF1R antibody), a B-raf inhibitor (e.g., vemurafenib, sorafenib, GDC-0879, PLX-4720), a PDGFR-alpha inhibitor (e.g., an anti-PDGFR-alpha. antibody), a PDGFR-.beta. inhibitor (e.g., an anti-PDGFR-beta. antibody or small molecule kinase inhibitor such as, e.g., imatinib mesylate or sunitinib malate), a PDGF ligand inhibitor (e.g., anti-PDGF-A, -B, -C, or -D antibody, aptamer, siRNA, etc.), a VEGF antagonist (e.g., a VEGF-Trap such as aflibercept, see, e.g., U.S. Pat. No. 7,087,411 (also referred to herein as a “VEGF-inhibiting fusion protein”), anti-VEGF antibody (e.g., bevacizumab), a small molecule kinase inhibitor of VEGF receptor (e.g., sunitinib, sorafenib or pazopanib)), a DLL4 antagonist (e.g., an anti-DLL4 antibody disclosed in US 2009/0142354 such as REGN421), an Ang2 antagonist (e.g., an anti-Ang2 antibody disclosed in US 2011/0027286 such as H1H685P), a FOLHI antagonist (e.g., an anti-FOLHI antibody), a STEAP1 or STEAP2 antagonist (e.g., an anti-STEAP1 antibody or an anti-STEAP2 antibody), a TMPRSS2 antagonist (e.g., an anti-TMPRSS2 antibody), a MSLN antagonist (e.g., an anti-MSLN antibody), a CA9 antagonist (e.g., an anti-CA9 antibody), a uroplakin antagonist (e.g., an anti-uroplakin [e.g., anti-UPK3A] antibody), a MUC16 antagonist (e.g., an anti-MUC16 antibody), a Tn antigen antagonist (e.g., an anti-Tn antibody), a CLEC12A antagonist (e.g., an anti-CLEC12A antibody), a TNFRSF17 antagonist (e.g., an anti-TNFRSF17 antibody), a LGR5 antagonist (e.g., an anti-LGR5 antibody), a monovalent CD20 antagonist (e.g., a monovalent anti-CD20 antibody such as rituximab), a PD-1 antibody, a PD-LI antibody, a CD3 antibody, a CTLA-4 antibody etc. Other agents that may be beneficially administered in combination with the UIR-T cells of the invention include, e.g., tamoxifen, aromatase inhibitors, and cytokine inhibitors, including small-molecule cytokine inhibitors and antibodies that bind to cytokines such as IL-1, IL-2, IL-3, IL-4, IL-5, IL-6, IL-8, IL-9, IL-11, IL-12, IL-13, IL-17, IL-18, or to their respective receptors.

The present invention includes compositions and therapeutic formulations comprising any of the immune cells, such as UIR-T cells, described herein in combination with one or more chemotherapeutic agents. Examples of chemotherapeutic agents include alkylating agents such as thiotepa and cyclosphosphamide (Cytoxan™); alkyl sulfonates such as busulfan, improsulfan and piposulfan; aziridines such as benzodopa, carboquone, meturedopa, and uredopa; ethylenimines and methy lamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; ctoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK™; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (Taxol™, Bristol-Myers Squibb Oncology, Princeton, N.J.) and docetaxel (Taxotere™; Aventis Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumours such as anti-estrogens including for example tamoxifen, raloxifene, aromatase inhibiting 4 (5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above.

The administration of any of the disclosed therapeutic agents may be carried out in any convenient manner, including by injection, transfusion, or implantation. The compositions described herein may be administered to a patient subcutaneously, intradermally, intratumourally, intranodally, intramedullary, intramuscularly, by intravenous (i.v.) injection, or intraperitoneally. In some embodiments, the disclosed compositions are administered by i.v. injection. The compositions may also be injected directly into a tumour, lymph node, or site of infection.

As will be appreciated by those skilled in the art, the above described cells and/or molecule will be administered to a subject in a therapeutically effective amount. The terms “effective amount” or “therapeutically effective amount” as used herein, refer to a sufficient amount of a therapeutic agent being administered which will relieve to some extent or prevent worsening of one or more of the symptoms of the disease or condition being treated. The result can be reduction or a prevention of progression of the signs, symptoms, or causes of a disease, or any other desired alteration of a biological system. For example, an “effective amount” for therapeutic uses is the amount of therapeutic agent required to provide a clinically significant decrease in disease symptoms without undue adverse side effects.

The term “therapeutically effective amount” includes, for example, a prophylactically effective amount. An “effective amount” of a therapeutic agent is an amount effective to achieve a desired pharmacologic effect or therapeutic improvement without undue adverse side effects. It is understood that “an effective amount” or “a therapeutically effective amount” can vary from subject to subject, due to variation in metabolism of the compound of any of age, weight, general condition of the subject, the condition being treated, the severity of the condition being treated, and the judgment of the prescribing physician.

It is considered well within the skill of the art for one to determine such therapeutically effective amounts by routine experimentation (including, but not limited to, a dose escalation clinical trial). An appropriate “effective amount” in any individual case may be determined using techniques, such as a dose escalation study.

Where more than one therapeutic agent is used in combination, a “therapeutically effective amount” of each therapeutic agent can refer to an amount of the therapeutic agent that would be therapeutically effective when used on its own, or may refer to a reduced amount that is therapeutically effective by virtue of its combination with one or more additional therapeutic agents.

### Methods of Treatment

The immune cells of or produced using the invention, e.g. UIR-T cells, are useful, inter alia, for the treatment, prevention and/or amelioration of a disease or disorder. For example, the UIR-T cells of the present invention are useful for the treatment of cancer, an infection, or an inflammatory disease. As another example, dendritic cells produced by a method of the invention can be used as a dendritic cell vaccine (see, for example, Datta et al., 2014) for treating, for example, a cancer, an infection (such as a bacterial or viral infection) or an autoimmune disease (such as diabetes). As a further example, NK cells, such as NK-UIR cells can be used to treat cancer (see, for example, Liu et al., 2021).

UIR cells may be used to treat primary and/or metastatic tumours arising in the brain and meninges, oropharynx, lung and bronchial tree, gastrointestinal tract, male and female reproductive tract, muscle, bone, skin and appendages, connective tissue, spleen, immune system, blood forming cells and bone marrow, liver and urinary tract, and special sensory organs such as the eye. In certain embodiments, UIR-cells of the invention are used to treat one or more of the following cancers: renal cell carcinoma, pancreatic carcinoma, head and neck cancer, prostate cancer, malignant gliomas, osteosarcoma, colorectal cancer, gastric cancer (e.g., gastric cancer with MET amplification), malignant mesothelioma, multiple myeloma, ovarian cancer, small cell lung cancer, non-small cell lung cancer, synovial sarcoma, thyroid cancer, breast cancer, melanoma, leukaemia, or lymphoma.

In an embodiment, the UIR-cells of the present invention are used to treat leukaemia, for example acute myeloid leukaemia, chronic myeloid leukaemia, acute lymphocytic leukaemia, or chronic lymphocytic leukaemia. In an embodiment, the leukaemia is acute myeloid leukaemia where low CD33+ blasts are dominant.

In another embodiment, the UIR-cells of the present invention are used to treat lymphoma, for example Hodgkin lymphoma or non-Hodgkin lymphoma. Non-Hodgkin lymphoma types include diffuse large B-cell lymphoma, anaplastic large-cell lymphoma, Burkitt lymphoma, lymphoblastic lymphoma, mantle cell lymphoma, or peripheral T cell lymphoma. In an embodiment, the lymphoma is diffuse large B cell lymphoma or non-Hodgkin lymphoma with low levels of CD19 and/or CD20.

Examples of antigens the armed universal immune receptor could bind include, but are not limited to, CD 19, CD20, ROR1, CD22carcinoembryonic antigen, alphafetoprotein, CA-125, 5T4, MUC-1, epithelial tumour antigen, prostate-specific antigen, melanoma-associated antigen, mutated p53, mutated ras, HER2/Neu, folate binding protein, HIV-1 envelope glycoprotein gp120, HIV-1 envelope glycoprotein gp41, GD2, CD123, CD33, CD138, CD23, CD30, CD56, c-Met, meothelin, GD3, HERV-K, IL-11Ra, k chain, 1 chain, CSPG4, ERBB2, EGFRvIII or VEGFR2.

In the context of the methods of treatment described herein, the immune cells, such as UIR-cells, may be administered as a monotherapy (i.e., as the only therapeutic agent) or in combination (combination therapy) with one or more additional therapeutic agents (examples of which are described elsewhere herein).

In one embodiment, the subject is at risk of developing a cancer (e.g., cancer). A subject is at risk if he or she has a higher risk of developing a cancer than a control population. The control population may include one or more subjects selected at random from the general population (e.g., matched by age, gender, race and/or ethnicity) who have not suffered from or have a family history of a cancer. A subject can be considered at risk for a cancer if a “risk factor” associated with a cancer is found to be associated with that subject. A risk factor can include any activity, trait, event or property associated with a given disorder, for example, through statistical or epidemiological studies on a population of subjects. A subject can thus be classified as being at risk for a cancer even if studies identifying the underlying risk factors did not include the subject specifically.

In one embodiment, the subject is at risk of developing a cancer and the cells, or compositions, are administered before or after the onset of symptoms of a cancer. In one embodiment, the cells, or compositions are administered before the onset of symptoms of a cancer. In one embodiment, the cells, or compositions are administered after the onset of symptoms of a cancer. In one embodiment, the cells, or compositions of the present invention is administered at a dose that alleviates or reduces one or more of the symptoms of a cancer in a subject at risk.

In an embodiment, the subject has been diagnosed as having, or is suspected of having a disease or disorder such as cancer, infection or an inflammatory disease. In an embodiment, the methods described herein comprise a step of diagnosing the subject as having or suspected of having a disease or disorder such as cancer, infection or an inflammatory disease.

Diagnosis as used herein refers to the determination that a subject or patient requires treatment with the immune cells of the invention. The type of disease or disorder diagnosed according to the methods described herein may be any type known in the art or described herein.

In an embodiment, the step of identifying or diagnosing a subject requiring treatment with the immune cells of the invention comprises the determination that the subject has cancer and may include assessment of one or more or all of:
blood profiling;
cell biopsy aspirates or tissue biopsy;
imaging such as computerized tomography (CT) scan, bone scan, magnetic resonance imaging (MRI), positron emission tomography (PET) scan, ultrasound and X-ray;
physical examination.

Examples of disease that can be treated with NK cells include, but are not limited to, cancers (e.g., melanoma, prostate cancer, breast cancer, and liver cancer) and infections, such as viral infections (e.g., infections by HSV, hepatitis viruses, human cytomegaloviruses, influenza viruses, flaviviruses, and HIV-1), bacterial infections (e.g., infections by Mycobacteria, Listeria, and Staphylococcus), and protozoan infections (e.g., infections by Plasmodium), and fungal infections (e.g., infections by Aspergillus).

Examples of inflammatory diseases include, but are not limited to, antibiotic-resistant microbial infection, idiopathic pulmonary fibrosis and Alzheimer's disease,

As will be apparent to the skilled person a “reduction” in a symptom of a cancer in a subject will be comparative to another subject who also suffers from a cancer but who has not received treatment with a method described herein. This does not necessarily require a side-by-side comparison of two subjects. Rather population data can be relied upon. For example, a population of subjects suffering from a cancer who have not received treatment with a method described herein (optionally, a population of similar subjects to the treated subject, e.g., age, weight, race) are assessed and the mean values are compared to results of a subject or population of subjects treated with a method described herein.

In one embodiment, the immune cells and methods of the present invention are used to improve survival of a subject suffering from a disease or disorder such as cancer, infection or an inflammatory disease. Where survival is contemplated, survival analysis can be performed using the Kaplan-Meier method. The Kaplan-Meier method estimates the survival function from life-time data and can be used to measure the fraction of patients living for a certain amount of time after treatment. A plot of the Kaplan-Meier method of the survival function is a series of horizontal steps of declining magnitude which, when a large enough sample is taken, approaches the true survival function for that population. The value of the survival function between successive distinct sampled observations (“clicks”) is assumed to be constant. An important advantage of the Kaplan-Meier curve is that the method can take into account “censored” data-losses from the sample before the final outcome is observed (for instance, if a patient withdraws from a study). On the plot, small vertical tick-marks indicate losses, where patient data has been censored. When no truncation or censoring occurs, the Kaplan-Meier curve is equivalent to the empirical distribution.

In statistics, the log-rank test (also known as the Mantel-Cox test) is a hypothesis test to compare the survival distributions of two groups of patients. It is a nonparametric test and appropriate to use when the data are right censored. It is widely used in clinical trials to establish the efficacy of new drugs compared to a control group when the measurement is the time to event. The log-rank test statistic compares estimates of the hazard functions of the two groups at each observed event time. It is constructed by computing the observed and expected number of events in one of the groups at each observed event time and then adding these to obtain an overall summary across all time points where there is an event. The log-rank statistic can be derived as the score test for the Cox proportional hazards model comparing two groups. It is therefore asymptotically equivalent to the likelihood ratio test statistic based on that model.

### EXAMPLES

### Example 1—Universal Immune Receptor

A SpyCatcher universal immune receptor was produced having, in order from N- to C-terminus, a CD8a leader, a Spy Catcher v003, a CD8a hinge, a CD8a transmembrane domain, a CD28z co-stimulatory domain and a CD3z intracellular signalling domain (SEQ ID NO: 1) encoded by the polynucleotide sequence provided as SEQ ID NO:2.

SEQ ID NO: 1—SpyCatcher universal immune receptor. The first underlined section is the CD8 leader, the second underlined section is Spy Catcher, the third underlined section is CD8a hinge, followed by the a CD8a transmembrane domain, the fourth underlined section is the CD8a transmembrane domain, followed by the CD28z co-stimulatory domain (excluding the C-terminal ID of this section), with the fifth underlined section being the CD3z intracellular signalling domain

MALPVTALLLPLALLLHAARPGSVTTLSGLSGEQGPSGDMTTEEDSATH
IKFSKRDEDGRELAGATMELRDSSGKTISTWISDGHVKDFYLYPGKYTF
VETAAPDGYEVATPIEFTVNEDGQVTVDGEATEGDAHTASTTTPAPRPP
TPAPTIASQPLSLRPEACRPAAGGAVHTRGLDFACDFWVLVVVGGVLAC
YSLLVTVAFIIFWVRSKRSRLLHSDYMNMTPRRPGPTRKHYQPYAPPRD
FAAYRSIDRVKFSRSADAPAYKQGQNQLYNELNLGRREEYDVLDKRRGR
DPEMGGKPRRKNPQEGLYNELQKDKMAEAYSEIGMKGERRRGKGHDGLY
QGLSTATKDTYDALHMQALPPR*

SEQ ID NO:2—Polynucleotide encoding the SpyCatcher universal immune receptor of SEQ ID NO:1.

atggccttaccagtgaccgccttgctcctgccgctggccttgctgctcc
acgccgccaggccgggatccGTGACAACACTGAGCGGACTGTCTGGCGA
GCAAGGCCCTTCTGGCGATATGACCACCGAAGAGGATAGCGCCACACAC
ATCAAGTTCAGCAAGCGCGACGAGGACGGCAGAGAACTTGCTGGCGCTA
CCATGGAACTGAGAGACAGCAGCGGCAAGACCATCAGCACCTGGATCTC
TGACGGCCACGTGAAGGACTTCTATCTGTACCCCGGCAAGTACACCTTC
GTGGAAACCGCCGCTCCTGACGGCTACGAAGTGGCCACACCTATCGAGT
TCACCGTGAACGAGGATGGCCAAGTGACCGTGGATGGCGAAGCTACAGA
AGGCGACGCCCATACAgctagcaccacgacgccagcgccgcgaccacca
acaccggcgcccaccatcgcgtcgcagcccctgtccctgcgcccagagg
cgtgccggccagcggggggggcgcagtgcacacgagggggctggacttc
gcctgtgatttttgggtgctggtggtggttggtggagtcctggcttgct
atagcttgctagtaacagtggcctttattattttctgggtgaggagtaa
gaggagcaggctcctgcacagtgactacatgaacatgactccccgccgc
cccgggcccacccgcaagcattaccagccctatgccccaccacgcgact
tcgcagcctatcgctccatcgatagagtgaagttcagcaggagcgcaga
cgcccccgcgtacaagcagggccagaaccagctctataacgagctcaat
ctaggacgaagagaggagtacgatgttttggacaagagacgtggccggg
accctgagatggggggaaagccgagaaggaagaaccctcaggaaggcct
gtacaatgaactgcagaaagataagatggcggaggcctacagtgagatt
gggatgaaaggcgagcgccggaggggcaaggggcacgatggcctttacc
agggtctcagtacagccaccaaggacacctacgacgcccttcacatgca
ggccctgccccctcgctaa

Furthermore, two SpyTag binding molecules were produced which bind Her2. One termed the standard Her2 binder comprising an N-terminal signal peptide, an antibody variable domain that binds Her2, an antibody constant domain, a linker and SpyTag (SEQ ID NO:3). The other termed short half life binder has an antibody constant domain which confers a shorter half life (SEQ ID NO:4). Following expression and processing the N-terminal signal peptide is removed such that the molecule administered lacks this peptide (SEQ ID NO's 5 and 6 respectively).

SEQ ID NO:3—Standard Her2 binder (SpyTagged heavy chain) with N-terminal signal sequence. The first underlined section is the N-terminal signal sequence, followed by the antibody variable domain that binds Her2, the second underlined section is the antibody constant domain, followed by a linker and the third underlined section is Spytag.

MAWSWVFLFFLSVTTGVHSEVQLVESGGGLVQPGGSLRLSCAASGFNIK
DTYIHWVRQAPGKGLEWVARIYPTNGYTRYADSVKGRFTISADTSKNTA
YLQMNSLRAEDTAVYYCSRWGGDGFYAMDYWGQGTLVTVSSASTKGPSV
FPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPE
VKFNWYVDGVEVHNAKTKPREEQYGSTYRVVSVLTVLHQDWLNGKEYKC
KVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQ
GNVFSCSVMHEALHNHYTQKSLSLSPGKGGGGSGGGGSRGVPHIVMVDA
YKRYK

SEQ ID NO:4—Short half-life Her2 variant (SpyTagged heavy chain) with N-terminal signal sequence. The first underlined section is the N-terminal signal sequence, followed by the antibody variable domain that binds Her2, the second underlined section is the short half life antibody constant domain, followed by a linker and the third underlined section is Spytag.

MAWSWVFLFFLSVTTGVHSEVQLVESGGGLVQPGGSLRLSCAASGFNIK
DTYIHWVRQAPGKGLEWVARIYPTNGYTRYADSVKGRFTISADTSKNTA
YLQMNSLRAEDTAVYYCSRWGGDGFYAMDYWGQGTLVTVSSASTKGPSV
FPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKT
HTCPPCPAPELLGGPSVFLFPPKPKDTLMASRTPEVTCVVVDVSHEDPE
VKFNWYVDGVEVHNAKTKPREEQYGSTYRVVSVLTVLHQDWLNGKEYKC
KVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQ
GNVFSCSVMHEALHNHYTQKSLSLSPGKGGGGSGGGGSRGVPHIVMVDA
YKRYK

SEQ ID NO:5—Standard Her2 binder (SpyTagged heavy chain) without N-terminal signal sequence (anti-Her2-ST).

EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYGSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGKGGGGSGGGGSRGVPHIVMVDAYKRYK

SEQ ID NO:6—Short half-life Her2 variant (SpyTagged heavy chain) without N-terminal signal sequence.

EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMASRTPEVTCVVVDVSHEDPEVKFNWYVDGVEVHNAKTKP
REEQYGSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGKGGGGSGGGGSRGVPHIVMVDAYKRYK

The DNA sequence relating to each binder is first synthesised de novo as a polynucleotide including the Spy Tag sequence, which was then cloned into a transfection grade, endotoxin-free plasmid (e.g. pAb20-hCL-1). This is then transiently transfected into a permissive cell line such as TunaCHO, which is then cultured for 14-days in DMEM/F12 and the binders are then purified using Protein A purification, size exclusion chromatography and ultra performance liquid chromatography. Confirmation of identity was performed using mass spectrometry. Additional binders have been developed and synthesised and are disclosed according to SEQ ID NOs: 8, 9, 10, 11, 12, 13, 14 15, 16, 17 and 18 herein.

### Example 2—Cell Transfection and Expansion

Preparations of either polymorphonuclear cells or enriched T cells will be activated with CD3/CD28 beads for 24 hours, then transduced with lentiviruses encoding for the SpyCatcher construct in the presence of retronectin or polybrene for a further 24 hours, whereupon 50 IU/mL of recombinant IL-2 would be added. The CD3/CD28 beads will be removed after 7 days of continuous culture, and the T cells will be cultured for a further 7 days. The transduction efficiency will be determined based on their expression of Spy Catcher on the cell surface, and T cell numbers and phenotype will be determined using a Coulter Counter and by multi-parameter flow cytometry.

### Example 3—Metronomic Dosing of UIR-T Cells

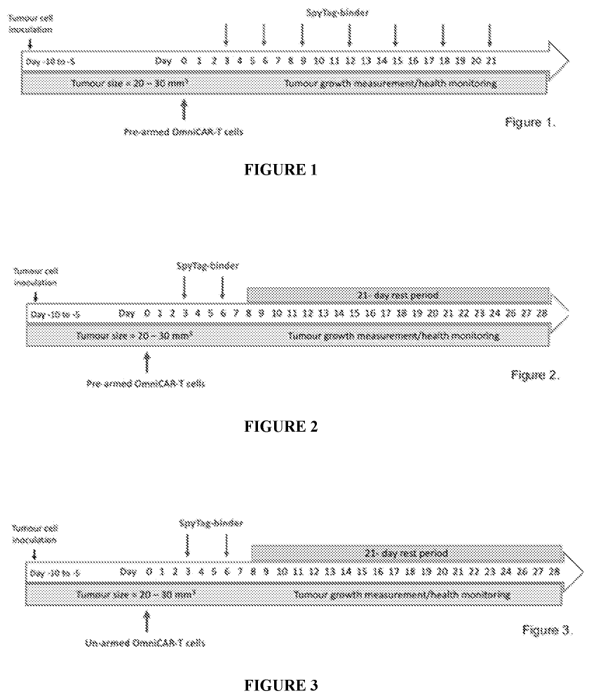

In order to determine the impact of metronomic dosing on UIR-T cell persistence and activity, immunodeficient NOD-SCID-gamma (NSG) mice will be injected with either 2-5×10⁶ MCF7 or SKBR3 breast cancer cells (via mammary fat pad or subcutaneous injection). Tumour growth will be measured every second day. After 5-10 days post-tumour injection or when tumour reaches 20-30 mm³, these mice will be preconditioned with 0.5-1 Gy of whole-body irradiation. On the same day, they will be intravenously injected with pre-armed UIR T cells (1×10⁷ UIR T cells in 200 uL PBS), with anti-Her2-ST (SpyTagged anti-Her 2 binder or the short-half-life version) every 3 days for 21 days (FIG. 1) at a concentration between 12.5 ug to 50 μg per mouse. Each dosing cycle lasts for 21 days.

The impact of intermittent dosing of ST-binders on persistence and UIR-T activity will also be assessed in a 28-day dosing protocol where pre-armed UIR T cells will be administered when tumour sizes reach 20-30 mm³ (Day 0) and anti-Her2-ST (or the short-half-life version) is administered twice in the first week (e.g. on days 3 and 6) at a concentration between 12.5 ug to 50 μg per mouse followed by a 21-day rest period (FIG. 2).

The impact of in vivo arming and intermittent dosing of ST-binders on UIR-T persistence and cytotoxicity will also be assessed in another 28-day dosing protocol where un-armed UIR T cells will be administered when tumour sizes reach 20-30 mm³ (Day 0) and anti-Her2-ST (or the short-half-life version) is administered thrice in the first week (e.g. on days 3 and 6) at a concentration between 12.5 ug to 50 μg per mouse followed by a 21-day rest period (FIG. 3).

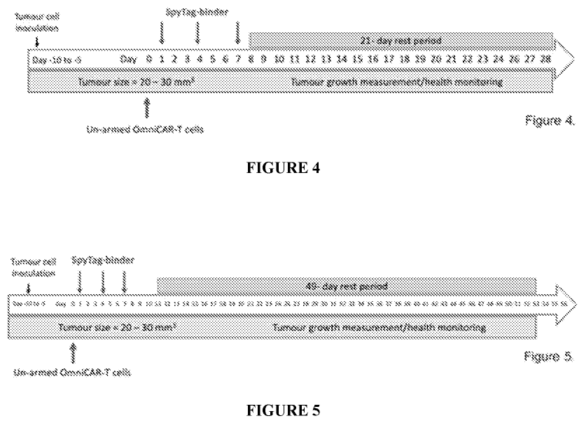

The impact of in vivo arming and intermittent dosing of ST-binders on UIR-T persistence and cytotoxicity will also be assessed in another 28-day dosing protocol where un-armed UIR T cells will be administered when tumour sizes reach 20-30 mm³ (Day 0) and anti-Her2-ST (or the short-half-life version) is administered thrice in the first week (e.g. on days 1, 4 and 7) at a concentration between 12.5 ug to 50 μg per mouse followed by a 21-day rest period (FIG. 4).

The impact of in vivo arming and intermittent dosing of ST-binders on UIR-T persistence and cytotoxicity will also be assessed in a longer 56-day dosing protocol where un-armed UIR T cells will be administered when tumour sizes reach 20-30 mm³ (Day 0) and anti-Her2-ST (or the short-half-life version) is administered thrice in the first week (e.g. on days 1, 4 and 7) at a concentration between 12.5 ug to 50 μg per mouse followed by a 49-day rest period (FIG. 5).

### Example 4—UIR-T Cell Anti-Tumour Efficacy and Dosing

### Materials and Methods

### Cell Lines and Mouse Models

Human breast cancer cell lines, MDA-MB231, were sourced from the ATCC and used to inoculate NOD-SCID gamma (NSG) mice. PCR analysis on each cell line was performed regularly to verify that lines were mycoplasma negative. All tumour cell lines were cultured in DMEM (Gibco, Life Technologies, Grand Island, New York, USA) supplemented with 10% heat-inactivated fetal bovine serum (FBS), 1 mM sodium pyruvate, 2 mM glutamine, 0.1 mM non-essential amino acids, 10 mM 4-(2-hydroxyethyl)-1-piperazineethanesulfonic acid (HEPES), 100 U/mL penicillin and 100 ug/mL streptomycin. Cells were grown in a humidified incubator at 37° C. with 10% CO₂. Other cell lines included retroviral and lentiviral packaging lines, HEK293gp, PA317 and GP+E86 that were obtained from American Type Culture Collection (ATCC, Manassas, VA, USA). GP+E86, PA317, HEK293T and HEK293gp were maintained in the same media as tumour cell media described above.

### 3rd Generation Lentivirus Production

3rd generation lentiviral envelope, packaging and transfer plasmids were used and were sourced from Addgene. Transfer plasmid was a modified variant of pULTRA plasmid. T175 flasks were pre-coated with poly-D-lysine (50 ug/mL) and washed prior to seeding of 23 million HEK293T packaging cells. 24 hours later, cells were transfected with 1:1:1:1 molar ratio of plasmids using Lipofectamine 3000 reagent as per manufacturer's instructions. Viral supernatant was collected on day 1, 2 and 3 post transfection, filtered (0.45 uM) and concentrated with the Lenti-X-Concentrator reagent from Takara Bio, before aliquots were frozen in −80° C.

### Lentiviral Transduction of Human PBMCs

Fresh donor human PBMCs were processed by diluting with 1:1 ratio of PBS, before being separated by Ficoll separation. White blood cells were separated from serum and red blood cells and collected before further red cell lysis. Post lysis, cells were activated with OKT3 (30 ng/mL), 600 IU of human IL-2 and complete RPMI (10% FCS, sodium pyruvate, glutamax, NEAA, HEPES and penicillin/streptomycin) media at a cell concentration of 1 million cells/mL for a minimum of 48 hours. Post activation, activated T cells were re-seeded at 1 million T cells/mL with 600 IU IL-2 before addition of 1MOI functional titre of lentivirus with lentiboost reagent (1:400). Cells were topped up with media and IL-2 and cultured for minimum of 72 hours.

### Flow Cytometry

Cells collected were first washed with FACS buffer and then stained with fluorophore-conjugated antibodies for 30 mins on ice. Cells were washed at least twice with FACS buffer before being resuspended in FACS buffer and count beads (20 uL, ˜20,000 beads). For intracellular or intranuclear stains, stained cells were further fixed and permeabilised using the BD Biosciences or eBioscience kit respectively as per manufacturer instructions. Fixed and permeabilised cells are then further stained fluorophore-conjugated antibodies for 30 mins at room temperature before washing with perm/wash buffer at least twice and final resuspension in perm/wash buffer with counting beads. Stained samples were acquired on BD FACSymphony, FACSFortessa or LSR (BD Biosciences). The number of target cell population was calculated by using: the number of input beads in the sample/bead events×events of targeted population.

### Coculture of CAR T Cells and Tumour Cells

For direct activation of human CAR T cells with OKT3 (1:1000), flat bottom 96 well plates were pre-coated for at least 1 hour at 37° C. For tumour cocultures, CAR T cells and tumour cells were cocultured in 200 uL supplemented RPMI media at varying E:T ratio with 50,000 tumour cells/well in flat bottom 96 well plates. Cells were co-cultured at 37° C. and 5% CO₂ for 24-72 hours as indicated.

### Incucyte Killing Assay

Tumour cells were first transduced to express target antigen linked to fluorescent markers GFP for HER2 or mCherry for EGFRVIII. Tumours were seeded in 384 well plate at 5000 cells/well overnight, prior to addition of varying ratios of CAR T cells to each well with the Caspase 3/7 dye provided by manufacturer at recommended concentrations. Plates were then added to the incucyte machine located inside a 5% CO₂, 37° C. incubator and imaged every 1-4 hours for a total of 24-72 hours. Images are analysed on the Incucyte analysis software.

### Quantification of Cytokine Production

Supernatants collected were analysed by cytometric bead array (CBA) assays (BD Biosciences). 12.5 uL of supernatant was transferred to V-bottom 96 well plates. Half serial dilutions of standards containing each cytokine tested were plated, with the highest concentration at 5000 pg/mL. As per manufacturer's instructions, a capture cocktail containing cytokine-specific beads of 0.25 uL beads/cytokine/well was made up to 12.5 uL/well (human) with BD bead buffer solution respectively and added to each well. The plate was then incubated for up to 1 hour at room temperature. The same volumes as beads for the PE detection antibody cocktail for mouse or human cytokines were made up and added to each well. Plates were again incubated for up to 1 hour at room temperature in the dark. Finally, cells were washed with provided wash buffer at least twice before resuspension in 75-100 uL of wash buffer and analysed on the BD FacsVerse (Becton Dickinson) and data processed on the FCAP software (BD biosciences).

### ‘Arming’ and ‘Pre-Arming’ of OmniCAR T Cells, Dose Scheduling of Antibody Binders

OmniCAR T cells that are unarmed do not have the targeting antibody binders bound to the spy Catcher receptor CAR and are not able to bind to tumour antigens and are therefore unable to activate and kill tumour cells. ‘Arming’ OmniCAR T cells by adding antibody binders at total 100 nM concentration to OmniCAR T cells in T cell media (<10^{e6}/mL) and incubating for around 30 minutes at 37 degrees Celsius in 5% CO2 will form functional spy Catcher: spyTAG receptors. ‘Pre-armed’ OmniCAR T cells are CAR T cells that are armed ex vivo before adoptive transfer or outside the body/mouse. Antibodies are washed off prior to adoptive transfer.

### Adoptive Transfer and Treatment of Tumour Bearing Mice

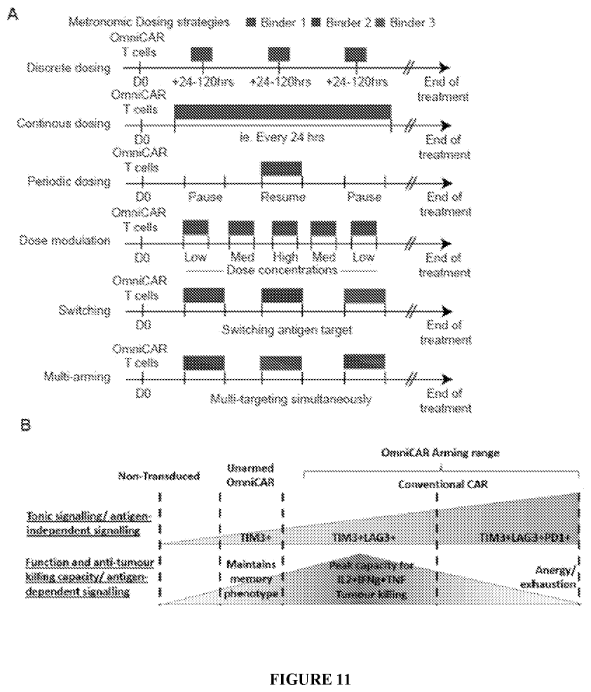

NSG mice were injected with 2-5×106 MDA-MB231 breast cancer cells transduced to express the human HER2 antigen sub-cutaneously. Post establishment of tumours, mice were then subject to preconditioning regiment of 0.5 gy total body irradiation, before the adoptive transfer of OmniCAR T cells supplemented with 4 doses of 25,000 IU of IL-2 per mouse every 24 hours. Binders were delivered intratumorally, intravenously or intraperitoneally based on metronomic dosing schedules (FIG. 11A).

### Analysis of Tumour-Infiltrating Immune Subsets

To examine tumour infiltrating CAR T cell phenotype, mice were euthanised and tumours or spleen were removed. Tumours were mechanically digested and enzymatically digested with DMEM supplemented with 1 mg/mL of collagenase type IV (Sigma-Aldrich) and 0.02 mg/mL DNAse (Sigma-Aldrich) for 30 minutes with constant shaking at 37° C. Tumour single cell suspensions are then filtered through 70 μm filters before aliquoting for flow cytometry staining. Spleens were also mechanically digested and filtered through 70 μm filters prior to staining.

### Results

### Dosing Regimen of Binders Regulates Functional UIR Expression In Vivo

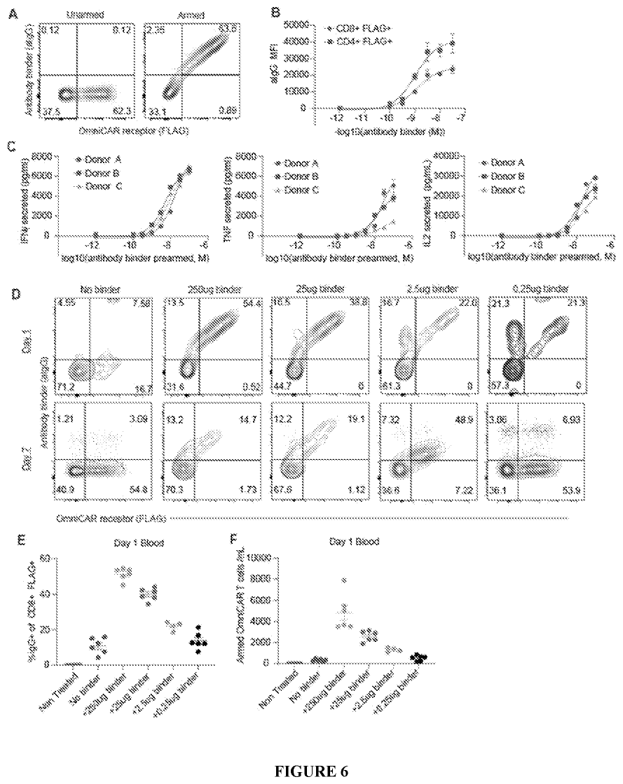

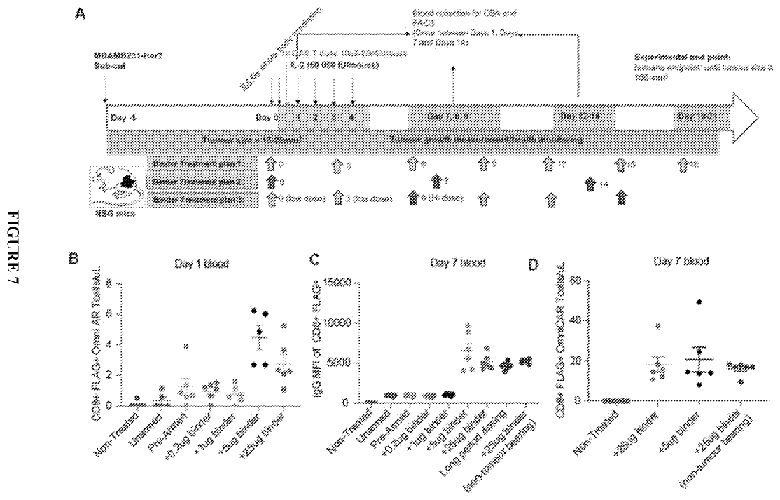

OmniCAR T cells were generated using 3rd generation lentivirus using the protocol described above, and transduction efficiency and expression of non-antigen specific OmniCAR receptors were detected using an anti-FLAG antibody (FIG. 6A). “Unarmed” OmniCAR T cells lack the antibody binder component and are so unable to bind to tumour antigens to elicit an anti-tumour response. The addition of an anti-human IgG antibody can detect the full-length antibody binders when they spontaneously form a complex with the OmniCAR receptors, which result in ‘Armed’ OmniCAR T cells that are fully functional against their respective target antigen (FIG. 6A).

Incubation with increasing concentration of antibody binders against the human HER2 antigen led to increased detection by anti-IgG, corresponding to increased expression of ‘Armed’ OmniCAR receptors on both CD8+ FLAG+ and CD4+ FLAG+CAR T cells (FIG. 6B). Correlating with increasing expression of armed receptors, OmniCAR T cells generated from 3 separate donors when co-cultured with breast cancer cell lines MDA-MB231 transduced with HER2 (MDA-MB231-HER2) were activated and secreted increasing concentrations of functional cytokines IFNγ, TNF and IL-2 (FIG. 6C). OmniCAR T cells can also be armed post adoptive transfer into NSG mice. For this purpose, 10-20 million OmniCAR T cells were transferred into NSG mice, and antibody binders were dosed at increasing concentrations every 3 days. Blood was collected at days 1 and 7 post transfer, and ‘armed’ FLAG+CAR T cells were detected at both time-points, with expression levels corresponding with increased binder dosing concentrations (FIG. 6D). At day 1 post therapy, higher dosage of binders led to increased detection of armed receptors (FIG. 6E) and early expansion and numbers of armed OmniCAR T cells (FIG. 6F). Similarly, the expression (MFI) of armed receptors at day 7 corresponded with increasing dosage of binders (FIG. 6D).

### Dosing Regimen of Binders Regulates T Cell Memory Phenotype, Expansion and Persistence In Vivo

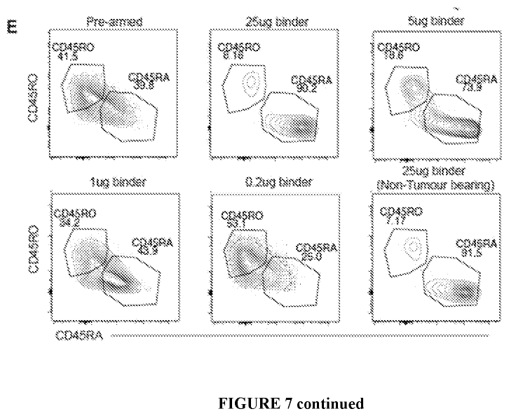

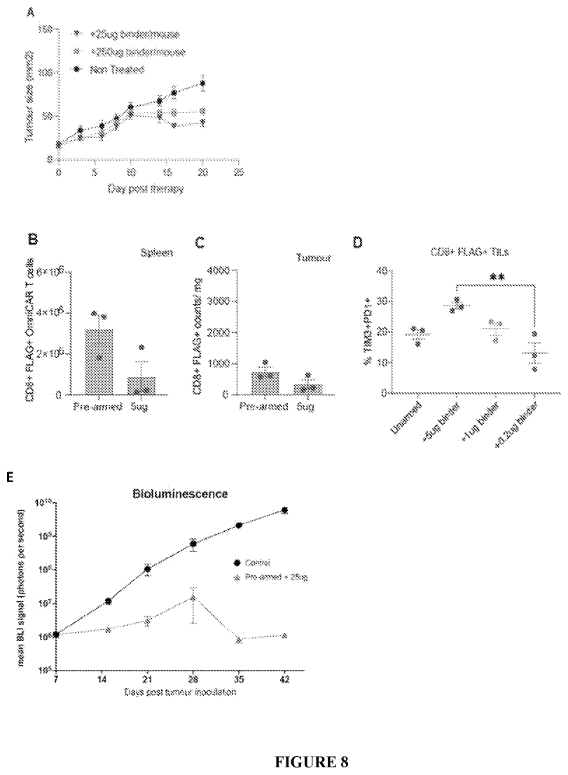

To further investigate the therapeutic efficacy of OmniCAR therapy, NSG mice were injected with human MDA-MB231-HER2 breast cancer cells. Once tumours were established, mice were treated with OmniCAR T cells and different doses and dosing regimens of anti-HER2 antibody binders (FIG. 7A). Like previous observations, higher dosage of antibody binders led to early expansion of CD8+ FLAG+ OmniCAR T cells in the periphery of mice (FIG. 7B). Dosing of binders >5 ug/mouse led to greater expression of ‘armed’ OmniCAR receptors, which was consistent with dosing of binders in either tumour or non-tumour bearing mice, or with long spaced-out periods between dosing of >1 week (FIG. 7C).

Modulating dose and dosing strategy could also modulate the memory phenotype of OmniCAR T cells in vivo, and this effect was observed in the context of tumour bearing and non-tumour bearing mice (FIG. 7D). Low concentrations of binders led to greater % of CD45RO+CD45RA-T effector memory subpopulations greater than pre-armed alone, while high concentrations of binders led to greater % of CD45RA+CD45RO-T central memory subpopulations which appears to be antigen-independent (FIG. 7D). Indeed, much of the expansion observed at day 7 of transferred OmniCAR T cells in the periphery in high dose treated groups was antigen-independent, indicating that the expansion of OmniCAR T cells is an intrinsic feature that is linked to modulating signalling from the OmniCAR receptor (FIG. 7E).

### Dosing Regimen of Binders Modulates Anti-Tumour Efficacy In Vivo

Consistent with observations of expansion and function, mice treated with pre-armed OmniCAR T cells and high dose of antibody binders could mediate anti-tumour effects compared to the non-treated mice, leading to reduced tumour sizes overall (FIG. 8A). At endpoint of the study, spleens and tumours were isolated from mice. As expected, high binder dose treatments led to reduced engraftment in spleen (FIG. 8B). Likewise, this pattern translated to the number of tumour infiltrating lymphocytes (TILs) detected in the tumours (FIG. 8C). Higher dosing of binders can also drive increased antigen-specific signalling due to increased functional OmniCAR receptor expression, leading to increased % of TIM3+PD1+CD8+ FLAG+CAR TILs (FIG. 8D).

The effect of the proposed regime was also tested in a model of acute myeloid leukemia (AML). Bioluminescence imaging was performed to determine tumour burden over time in the mouse model of AML. NSG mice were give 5 million KG-1 cells and the animals were either left untreated (control) or they were given pre-armed OmniCAR-T cells and 25 ug of CD33 and CLL-1 binder on days 3, 6 and 9 post CAR-T transfer. A significant effect on tumour growth was observed in mice treated with pre-armed OmniCAR-T cells and 25 ug of CD33 and CLL-1 binder on days 3, 6 and 9 post CAR-T transfer, when compared to the control group (FIG. 8E).

### Dosing Regimen or Specific Design of Binders Regulates OmniCAR Antigen-Independent Signaling or Antigen-Dependent Signalling

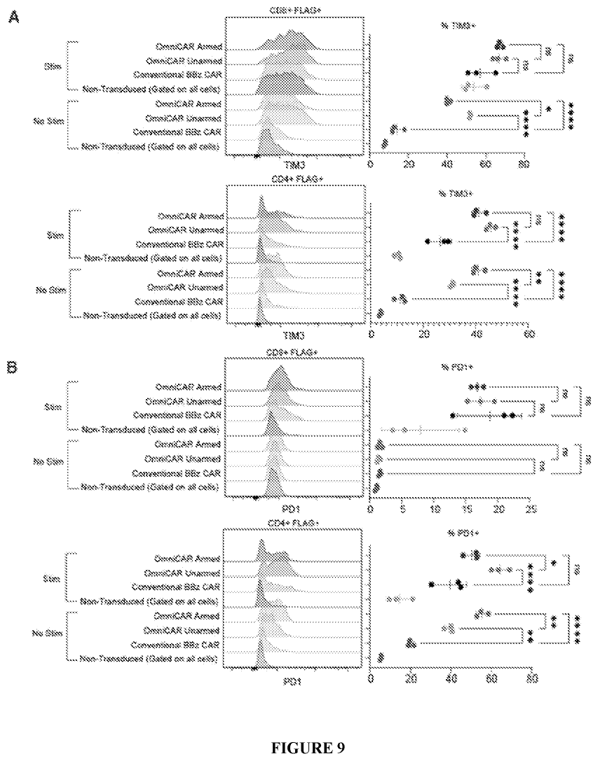

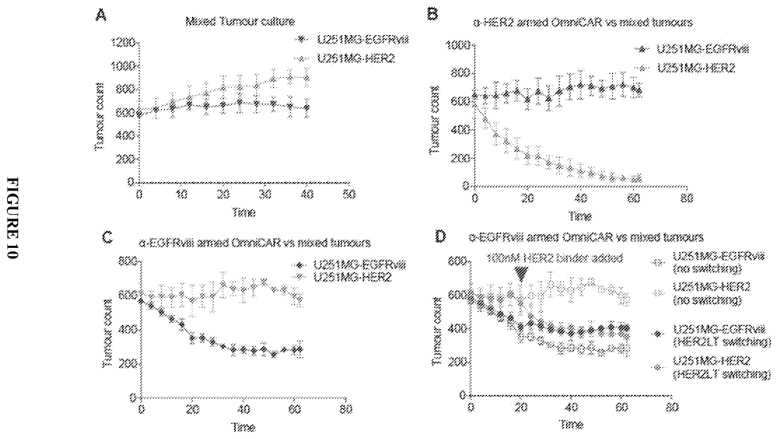

The intrinsic capacity of OmniCAR to drive superior proliferation of CAR T cells post adoptive transfer compared to conventional CAR T or non-transduced T cells was previously shown to be antigen-independent (FIG. 7D). To investigate the potential mechanisms involved, OmniCAR T cells were directly compared to non-transduced or conventional CAR T cells in vitro for levels of expression of activation, proliferation, or checkpoint T cell markers. After co-culture for >72 hours without stimulation in the presence of IL-2, both CD8 and CD4, unarmed or armed OmniCAR T cells respectively, upregulated expression of the activation marker TIM3 when compared to non-transduced or conventional CAR T cells (FIG. 9A).

Importantly both CD8 and CD4 armed OmniCAR T cells expressed higher levels of Tim3 compared to their unarmed counterparts (FIG. 9A). When stimulated with OKT3 (αCD3) for >72 hours, all CD8+ groups upregulated Tim3 to a similar level, although both unarmed or armed OmniCAR T cells had higher expression overall (FIG. 9A). Interestingly, both unarmed and armed CD4+ OmniCAR T cells had significantly higher Tim3 expression compared to conventional CAR T cells, indicating a greater level of activation when stimulated with OKT3 (FIG. 9A). While a second late checkpoint receptor, PD-1 was not expressed highly in vitro without stimulation, it was upregulated with stimulation but not by arming in CD8+ fractions (FIG. 9B). Expression of PD-1 was pronounced in the CD4+ fractions however, with unarmed and armed CD4+ OmniCAR T cells expressing higher levels of PD-1, with arming in unstimulated cells leading to even higher PD-1 expression (FIG. 9B). PD-1 continues to be elevated in stimulated CD4+ OmniCAR T cells, although armed OmniCAR T cells had reduced PD-1 compared to unarmed, indicating possible reduced signalling from armed OmniCAR receptors in CD4+ T cells (FIG. 9B).

Taken together, the unarmed or armed OmniCAR receptor has unique signalling effects on unstimulated or stimulated CD8 or CD4 fractions respectively, highlighting an application for differentially modulating the different fractions of the OmniCAR T cell product post adoptive transfer. A complex interplay between CD8 and CD4 fractions may drive the superior proliferation of transferred OmniCAR T cells.

### Metronomic Dosing to Target Multiple Tumour Antigens Sequentially or Simultaneously

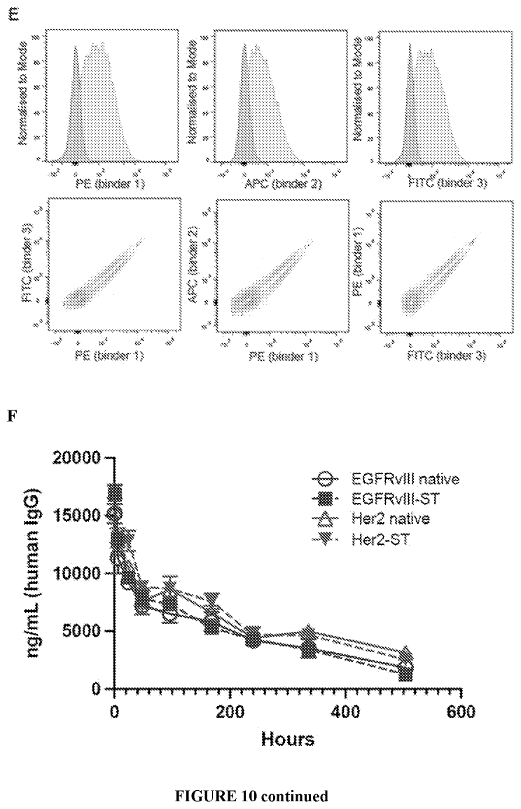

An advantage of the OmniCAR platform is that multiple tumour antigens can be targeted through sequential or simultaneous dosing of different antibody binders. Human glioblastoma cell line U251MG was modified to express either the human HER2 or mutant EGFRvIII receptors separately. In a mixed tumour co-culture, both U251MG-HER2 and U251MG-EGFRvIII had stable growth kinetics (FIG. 10A). When OmniCAR T cells were armed against the human HER2 antigen alone, only the HER2 expressing U251MG-HER2 cells were eliminated in a mixed tumour co-culture (FIG. 10B). Similarly, when OmniCAR T cells were armed against the mutant EGFRVIII antigen alone, only the EGFRvIII expressing U251MG-EGFRVIII cells were eliminated in a mixed tumour co-culture (FIG. 10C). Finally, to demonstrate antigen switching to a different target, EGFRvIII targeting OmniCAR T cells were co-cultured in a mixed tumour assay, and 100 nM of HER2 binder was added 20 hours later (FIG. 10D). Until HER2 binder was added, only EGFRvIII expressing tumours were eliminated, but CAR T cell killing switched to HER2 expressing tumours post addition of the HER2 binders, indicating rapid and efficient targeting of antigens sequentially (FIG. 10D). To target multiple tumour antigens simultaneously, OmniCAR T cells can be armed with more than one antibody binder at once, with equivalent levels of expression and co-expression of 3 different binders or more (FIG. 10E). Lastly, the presence of HER2 and EGFRVIII antibody binders can be detected in the sera of mice after 2 weeks following their administration (FIG. 10F).

Chimeric antigen receptor (CAR) T cell therapy has had widespread success in treating haematological malignancies and are undergoing clinical trials to treat multiple solid tumours. However, there are several challenges related to unique toxicities and subsequent relapses that need to be addressed. Universal immune receptors is a rapidly emerging arm form of adoptive immunotherapy that have the potential to address these challenges through increased safety and reduced side effects (switching on/off CAR responses post-infusion) and, targeting multiple tumour antigens to overcome tumour escape mechanisms (such as antigen loss or antigen heterogeneity) which lead to tumour relapse. Additionally, universal CAR T cells could potentially have much greater versatility, reduced cost and off the shelf utility potential compared to conventional CAR therapies currently in the clinic. Therefore, metronomic dosing strategies, highlighted in FIG. 11A, can endow OmniCAR T cells with the capacity to not only regulate expression of antigen specific functional UIR expression in vitro and in vivo, but also the capacity to regulate T cell memory differentiation, expansion and persistence in vivo.

By modulating dosing regiments temporally, through discrete, continuous or periodic dosing, or by modulating dose concentrations (FIG. 11A), it is possible to directly or indirectly regulate the strength of antigen-independent tonic signalling and antigen dependent CAR signalling leading to maintenance of optimal T cell memory subsets, T cell activation and anti-tumour function (FIG. 11B). Intrinsic to the OmniCAR platform is the ability to modulate antigen independent tonic signalling that drives improved safety, anti-tumour activity and T cell expansion at levels greater than conventional CAR T cells which have a rigid architecture and thus fixed levels of tonic signalling (FIG. 11B). Multiple antigens can also be targeted with the ability to sequentially switch rapidly and efficiently between antigen targets or arming with more than one antigen binder to target multiple tumour antigens simultaneously (FIG. 10A-E). In conclusion, these findings support metronomic dosing as a strategy that synergistically combines with the flexibility of the OmniCAR UIR platform to fine-tune the anti-tumour therapeutic response post adoptive transfer.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Berg et al. (1998) Transplant Proc. 30:3975-3977.
Datta et al. (2014) Yale J Biol Med 87:491-518.
Garland et al. (1999) J. Immunol Meth. 227:53-63.
Ghosh et al. (1991) Glycobiology 5:505-510.
Haanen et al. (1999) J. Exp. Med. 190:1319-1328.
Lui et al. (2021) J Hemat Oncol 14:7.
Rosenberg et al. (1988) New Eng. J. of Med 319:1676.
Sabado et al. (2017) Cell Res 27:74-95.
Smith et al. (2015) Clinical & Translational Immunology 4: e31.
Veggiani et al. (2016) PNAS 113:1202-1207.

1. A method of treating a disease in a subject that would benefit from an immune cell therapy, the method comprising
i) administering immune cells comprising a universal immune receptor to the subject, wherein the universal immune receptor may or may not be covalently bound to a molecule which comprises a domain which binds an antigen associated with the disease,
ii) administering the molecule to the subject at least twice within seven days following step i),
iii) at least 21 days following step ii) analysing the subject for responsiveness to the treatment, and
iv) repeating steps i) and ii) if the subject has been responsive to the treatment but the disease is still detectable.

2. A method of stimulating a universal immune receptor mediated immune response to a tumour in a subject, the method comprising
i) administering immune cells comprising a universal immune receptor to the subject, wherein the universal immune receptor may or may not be covalently bound to a molecule which comprises a domain which binds an antigen associated with the tumour,
ii) administering the molecule to the subject at least twice within seven days following step i),
iii) at least about 21 days following step ii) analysing the subject for responsiveness to the treatment, and
iv) repeating steps i) and ii) if the subject has been responsive to the treatment but the tumour is still detectable.

3. The method of claim 1 or 2 wherein the molecule is not bound to the universal immune receptor in step i).

4. The method of claim 1 or 2, wherein the molecule is bound to the universal immune receptor in step i).

5. The method according to any one of claims 1 to 4, wherein in step ii) the molecule is administered twice.

6. The method of claim 5, wherein the molecule is administered on days 3 and 6 following step i).

7. The method according to any one of claims 1 to 4, wherein the molecule is administered three times following step i).

8. The method of claim 7, wherein the molecule is administered on days 1, 4 and 6 following step i).

9. The method according to any one of claims 1 to 8, wherein between 21 days and 49 days following step i), the subject is analysed for responsiveness to the treatment.

10. The method according to any one of claims 1 to 9, wherein the treatment comprises administering the molecule to the subject at least once prior to step i) and at least twice within seven days following step i).

11. The method according to any one of claims 1 to 10, wherein the treatment comprises administering the molecule to the subject twice prior to step i) and at least twice within seven days following step i).

12. The method according to any one of claims 1 to 11, wherein the molecule is administered at different doses, preferably at a dose of between about 0.25 mg/m²-2.0 mg/m², a dose of between about 5 mg/m²-25 mg/m², and a dose of between about 50 mg/m²-100 mg/m².

13. The method according to any one of claims 1 to 12, wherein step iv) comprises administering a universal immune receptor which may or may not be covalently bound to a molecule which comprises a domain which binds the same antigen as the molecule of step i).

14. The method according to any one of claims 1 to 12, wherein step iv) comprises administering a universal immune receptor which may or may not be covalently bound to a molecule which comprises a domain which binds a different antigen as the molecule of step i).

15. The method according to any one of claims 1 to 14, wherein the molecule comprises a domain which binds more than one antigen associated with the disease, preferably two antigens associated with the disease.

16. A method of treating a disease in a subject that would benefit from an immune cell therapy the method comprising
i) administering immune cells comprising a universal immune receptor, wherein the universal immune receptor may or may not be covalently bound to a molecule which comprises a domain which binds an antigen associated with the disease,
ii) administering the molecule to the subject every two or three days for between 14 days and 28 days following step i), and
iii) repeating steps i) and ii) if the subject has been responsive to the treatment but the disease is still detectable.

17. A method of stimulating a universal immune receptor mediated immune response to a tumour in a subject, the method comprising
i) administering immune cells comprising a universal immune receptor to the subject, wherein the universal immune receptor may or may not be covalently bound to a molecule which comprises a domain which binds an antigen associated with the tumour,
ii) administering the molecule to the subject every two or three days for between about 14 days and about 28 days following step i), and
iii) repeating steps i) and ii) if the subject has been responsive to the treatment but the tumour is still detectable.

18. The method of claim 16 or 17, wherein the molecule is not bound to the universal immune receptor in step i).

19. The method of claim 16 or 17, wherein the molecule is bound the universal immune receptor in step i).

20. The method of any one of claims 16 to 19, wherein the molecule is administered every three days following step i).

21. The method of claim 20, wherein the molecule is administered every three days for 21 days following step i).

22. The method of any one of claims 16 to 21, wherein the molecule is administered to the subject at least once prior to step i) and every two or three days for between about 14 days and about 28 days following step i).

23. The method of any one of claims 16 to 22, wherein the molecule is administered to the subject twice prior to step i) and every two or three days for between about 14 days and about 28 days following step i).

24. The method of claim 2 or 17, wherein stimulating a universal immune receptor mediated immune response to a tumour comprises increasing cytokine levels in the subject, preferably increasing levels of one or more or all of interferon-γ (IFN-γ), tumour necrosis factor (TNF) and interleukin-2 (IL-2).

25. The method of any one of claims 16 to 24, wherein step iii) comprises administering a universal immune receptor which may or may not be covalently bound to a molecule which comprises a domain which binds the same antigen as the molecule of step i).

26. The method of any one of claims 16 to 24, wherein step iii) comprises administering a universal immune receptor which may or may not be covalently bound to a molecule which comprises a domain and which binds a different antigen as the molecule of step i).

27. The method of any one of claims 16 to 26, wherein the molecule comprises a domain which binds more than one antigen associated with the disease, preferably two antigens associated with the disease.

28. The method of any one of claims 1 to 27, wherein the treatment increases survival in the subject when compared to a subject not receiving the treatment.

29. The method of any one of claims 1 to 28, further comprising a step of diagnosing the subject as having or suspected of having a disease or cancer.

30. The method of any one of claims 1 to 29, further comprising the administration of an additional therapeutic agent, optionally selected from the group consisting of chemotherapy, radiotherapy, surgery, bone marrow transplant, drug therapy, cryoablation or radiofrequency ablation.

31. The method of any one of claims 1 to 30, wherein the universal immune receptor comprises a SpyCatcher or a SpyTag extracellular binding domain bound to an extracellular hinge region, which is in turn bound to a transmembrane domain which is in turn bound to an immune cell receptor intracellular signaling domain.

32. The method of claim 31, wherein the universal immune receptor intracellular signaling domain further comprises a costimulatory molecule.

33. The method of claim 31 or claim 32, wherein the SpyCatcher extracellular binding domain is bound to the extracellular hinge domain.

34. The method of claim 31 or claim 32, wherein the SpyTag extracellular binding domain is bound to the extracellular hinge domain.

35. The method of any one of claims 1 to 34, wherein the molecule comprises a Spy Catcher or a Spy Tag and the domain.

36. The method of any one of claims 1 to 35, wherein the domain is a selected from the group consisting of an antibody, an antibody fragment, a scFv, a protein scaffold, a peptide, a ligand, an oligonucleotide, an aptamer, a labelling agent, a tumour antigen, a self-antigen, a viral antigen, and any combination thereof.

37. The method of claim 35, wherein the molecule comprises Spy Tag.

38. The method of claim 35, wherein the molecule comprises SpyCatcher.

39. The method of any one of claims 1 to 11 or 13 to 38, wherein the molecule is administered at a dose of between about 0.25 mg/m²-2.0 mg/m², a dose of between about 5 mg/m²-25 mg/m², or a dose of between about 50 mg/m²-100 mg/m².

40. The method of any one of claims 1 to 39, wherein the immune cells are T cells, NK cells, dendritic cells, myeloid cells, macrophages, stem cells or a combination thereof.

41. The method of claim 40, wherein the T cells are CD3+ T cells.

42. The method of claim 40 or claim 41, wherein the T cells are cytotoxic T cells, gamma delta T cells, T regulatory cells or iNKT cells.

43. The method of claim 40, wherein between about 10% and about 50% of the immune cells are CD8+ cells.

44. The method of claim 40, wherein the method provides for an enrichment of CD45RO+CD45RA− T effector memory cells and/or an enrichment of CD45RA+CD45RO− T central memory cells.

45. The method of any one of claims 1 to 44, wherein the cells are autologous cells.

46. The method of any one of claims 1, 3 to 16 or 18 to 45, wherein the disease is cancer, an infection or an inflammatory disease.

47. The method of claim 46, wherein the cancer is renal cell carcinoma, pancreatic carcinoma, head and neck cancer, prostate cancer, glioblastoma, malignant gliomas, osteosarcoma, colorectal cancer, gastric cancer, malignant mesothelioma, multiple myeloma, ovarian cancer, small cell lung cancer, non-small cell lung cancer, synovial sarcoma, thyroid cancer, breast cancer, melanoma, leukaemia, acute myeloid leukaemia (AML) or lymphoma.

48. The method of any one of claims 1 to 47, wherein the subject is a mammal.

49. The method of any one of claims 1 to 48, wherein the subject is a human.

50. Use of immune cells comprising a universal immune receptor for the manufacture of a medicament for treating a disease in a subject that would benefit from an immune cell therapy, wherein the universal immune receptor may or may not be covalently bound to a molecule which comprises a domain which binds an antigen associated with the disease, wherein the molecule will be administered to the subject at least twice within seven days following administration of the cells, wherein at least 21 days following the seven days the subject will be analysed for responsiveness to the treatment, and wherein the treatment is repeated if the subject has been responsive to the treatment but the disease is still detectable.

51. Use of immune cells comprising a universal immune receptor for the manufacture of a medicament for stimulating a universal immune receptor mediated immune response to a tumour in a subject, wherein the universal immune receptor may or may not be covalently bound to a molecule which comprises a domain which binds an antigen associated with the tumour, wherein the molecule will be administered to the subject at least twice within seven days following administration of the cells, wherein at least 21 days following the seven days the subject will be analysed for responsiveness to the treatment, and wherein the treatment is repeated if the subject has been responsive to the treatment but the tumour is still detectable.

52. Use of immune cells comprising a universal immune receptor for the manufacture of a medicament for treating a disease in a subject that would benefit from an immune cell therapy, wherein the universal immune receptor may or may not be covalently bound to a molecule which comprises a domain which binds an antigen associated with the disease, wherein the molecule will be administered to the subject every two or three days for between 14 days and 28 days following administration of the cells, and wherein the treatment is repeated if the subject has been responsive to the treatment but the disease is still detectable.

53. Use of immune cells comprising a universal immune receptor for the manufacture of a medicament for stimulating a universal immune receptor mediated immune response to a tumour in a subject, wherein the universal immune receptor may or may not be covalently bound to a molecule which comprises a domain which binds an antigen associated with the tumour, wherein the molecule will be administered to the subject every two or three days for between 14 days and 28 days following administration of the cells, and wherein the treatment is repeated if the subject has been responsive to the treatment but the tumour is still detectable.

54. A substantially purified and/or recombinant polypeptide comprising a sequence of amino acids provided as SEQ ID NO:5 or SEQ ID NO:6, or a sequence of amino acids at least 90% identical to one or both of SEQ ID NO:5 and SEQ ID NO:6, wherein the polypeptide is capable of covalently binding to a protein comprising SpyCatcher and binding a HER2 receptor on cancer cell.

55. A substantially purified and/or recombinant polypeptide comprising a sequence of amino acids provided as SEQ ID NO: 7 and/or SEQ ID NO: 10, or provided as SEQ ID NO: 8 and/or SEQ ID NO:9 or a sequence of amino acids at least 90% identical thereto, wherein the polypeptide is capable of covalently binding to a protein comprising SpyCatcher and binding an EGFRVIII receptor on a cancer cell.

56. A substantially purified and/or recombinant polypeptide comprising a sequence of amino acids provided as SEQ ID NO: 11 and/or SEQ ID NO: 14, or provided as SEQ ID NO: 12 and/or SEQ ID NO:13 or a sequence of amino acids at least 90% identical thereto, wherein the polypeptide is capable of covalently binding to a protein comprising SpyCatcher and binding an IL-13Ra2 receptor on a cancer cell.

57. A substantially purified and/or recombinant polypeptide comprising a sequence of amino acids provided as SEQ ID NO:15 and/or SEQ ID NO: 16, or a sequence of amino acids at least 90% identical thereto, wherein the polypeptide is capable of covalently binding to a protein comprising SpyCatcher and binding an CD33 receptor on a cancer cell.

58. A substantially purified and/or recombinant polypeptide comprising a sequence of amino acids provided as SEQ ID NO: 17 and/or SEQ ID NO:18, or a sequence of amino acids at least 90% identical thereto, wherein the polypeptide is capable of covalently binding to a protein comprising SpyCatcher and binding an C-type lectin-like (CLL1) receptor on a cancer cell.

59. An isolated and/or exogenous polynucleotide encoding the polypeptide of any one of claims 54 to 58.

60. A vector comprising the polynucleotide of claim 59.

61. An isolated transgenic cell comprising a polynucleotide of claim 59 and/or a vector of claim 60.

62. A method of producing a polypeptide of claim 59, the method comprising culturing cells of claim 61, and purifying the polypeptide from the cells or culture medium.

63. A pharmaceutical composition comprising immune cells comprising a universal immune receptor which may or may not be covalently bound to a molecule which comprises a domain which binds an antigen associated with a disease, wherein the domain comprises one or more or all of SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO: 13, SEQ ID NO:14, SEQ ID NO:15, SEQ ID NO:16, SEQ ID NO:17, SEQ ID NO: 18, or a sequence of amino acids at least 90% identical thereto.
